(19) United States
(12) Patent Application Publication
(10) Pub. No.: US 2016/0060196 A1
(43) Pub. Date: Mar. 3, 2016

(54) CRYSTALLIZED HYDROQUINONE AND METHODS OF MAKING

(71) Applicant: Eastman Chemical Company, Kingsport, TN (US)

(72) Inventors: Venkateswarlu Bhamidi, Kingsport, TN (US); Kyle Irion, Johnson City, TN (US); Michael Joseph Stoltz, Kingsport, TN (US); Richard S. Lott, Johnson City, TN (US)

(73) Assignee: Eastman Chemical Company, Kingsport, TN (US)

(21) Appl. No.: 14/835,214

(22) Filed: Aug. 25, 2015

Related U.S. Application Data
(60) Provisional application No. 62/042,100, filed on Aug. 26, 2014.

(51) Int. Cl. C07C 37/84; B65B 63/08; C07C 39/08

(52) U.S. Cl. CPC C07C 37/84; C07C 39/08; B65B 63/08

(57) ABSTRACT

Crystallized hydroquinone particles and methods for making the same are provided. Cooling profiles applied during crystallization of the hydroquinone from solution may be optimized to provide crystallized particles having suitable properties and that exhibit reduced agglomeration tendencies, even after long periods of time and/or transportation over long distances.

### CROSS-REFERENCE TO RELATED APPLICATIONS

This application claims priority to U.S. Provisional Patent Application Ser. No. 62/042,100, filed on Aug. 26, 2014, the disclosure of which is incorporated herein by reference in its entirety.

### BACKGROUND

1. Technical Field

One or more embodiments of the present invention relate to production of dihydroxybenzenes including, for example, hydroquinone. In particular, one or more embodiments of the present invention relate to the production of crystallized hydroquinone particles.

2. Description of Related Art

Hydroquinone is utilized for applications in a wide variety of fields, including, for example, photography, cosmetics, and personal care, as well as a starting material or intermediate in the synthesis of many chemical and pharmaceutical compounds. Additionally, hydroquinone is utilized in the field of polymerization, usually as a stabilizing agent or polymerization inhibitor, but also, at times, as a monomer, such as, for example, in polymerization of liquid crystalline polymers. Hydroquinone is typically produced in the form of needlelike crystalline particles, which may be susceptible to agglomeration or caking during transport, loading, and/or unloading.

Thus, a need exists for hydroquinone particles, and processes for making such particles, that exhibit a reduced tendency to cake during transport, including loading and unloading operations. Desirably, such particles could be produced with conventional processing equipment at the same or lower cycle time than is typically required, while providing hydroquinone particles with enhanced flowability and reduced agglomeration tendencies, even after the particles have been stored for long periods of time and/or transported over long distances.

### BRIEF DESCRIPTION OF THE DRAWINGS

Various embodiments of the present invention are described in detail below with reference to the attached drawing Figures, wherein:

FIG. 1 provides a schematic depiction of a portion of a hydroquinone production facility configured according to one embodiment of the present invention, particularly illustrating the recovery portion of the facility;

FIG. 2 is a flow chart representing certain steps involved in a method for crystallizing hydroquinone according to one embodiment of the present invention;

FIG. 3 is a graphical depiction of several exemplary cooling profiles used to cool and crystallize a hydroquinone-containing solution according to various embodiments of the present invention;

FIG. 4 is a graphical depiction of several cooling profiles used to cool and crystallize a hydroquinone-containing solution as described in Example 1;

FIG. 5 provides several exemplary crystallization cooling profiles modeled in Example 2;

FIG. 6 is a graphical depiction of a comparative cooling profile used to cool and crystallize a hydroquinone-containing solution as described in Example 3;

FIG. 7 is a graphical depiction of an inventive cooling profile used to cool and crystallize a hydroquinone-containing solution as described in Example 3;

FIG. 8 is a graphical depiction of another inventive cooling profile used to cool and crystallize a hydroquinone-containing solution as described in Example 3;

FIG. 9 is a graphical depiction of yet another inventive cooling profile used to cool and crystallize a hydroquinone-containing solution as described in Example 3;

FIG. 10 provides a graphical comparison of the particle size distribution for the hydroquinone particles crystallized according to the cooling profile provided in FIG. 6, in the upper portion, and the particle size distribution for hydroquinone particles crystallized according to the cooling profile shown in FIG. 7, in the lower portion;

FIG. 11 provides a graphical comparison of the particle size distribution for the hydroquinone particles crystallized according to the cooling profile provided in FIG. 6, in the upper portion, and the particle size distribution for hydroquinone particles crystallized according to the cooling profile shown in FIG. 8, in the lower portion;

FIG. 12 provides a graphical comparison of the particle size distribution for the hydroquinone particles crystallized according to the cooling profile provided in FIG. 6, in the upper portion, and the particle size distribution for hydroquinone particles crystallized according to the cooling profile shown in FIG. 9, in the lower portion;

FIG. 13 provides a graphical comparison of the particle size distribution for the hydroquinone particles crystallized according to the inventive cooling profile provided in FIG. 9, in the upper portion, and the particle size distribution for hydroquinone particles crystallized according to another comparative method, in the lower portion;

FIG. 14 is a graphical depiction of the aspect ratio distribution of Comparative Sample E measured in Example 5;

FIG. 15 is a graphical depiction of the aspect ratio distribution of Comparative Sample F measured in Example 5; and

FIG. 16 is a graphical depiction of the aspect ratio distribution of Inventive Sample 5 measured in Example 5.

### DETAILED DESCRIPTION

Several embodiments of the present invention may relate to methods of making hydroquinone and, in particular, to methods of recovering hydroquinone from a hydroquinone-containing solution through crystallization and subsequent recovery of the crystallized particles. Hydroquinone particles produced as described herein may exhibit enhanced properties, such as increased particle size and better flowability, which may facilitate preservation of the crystallized material during transportation and storage.

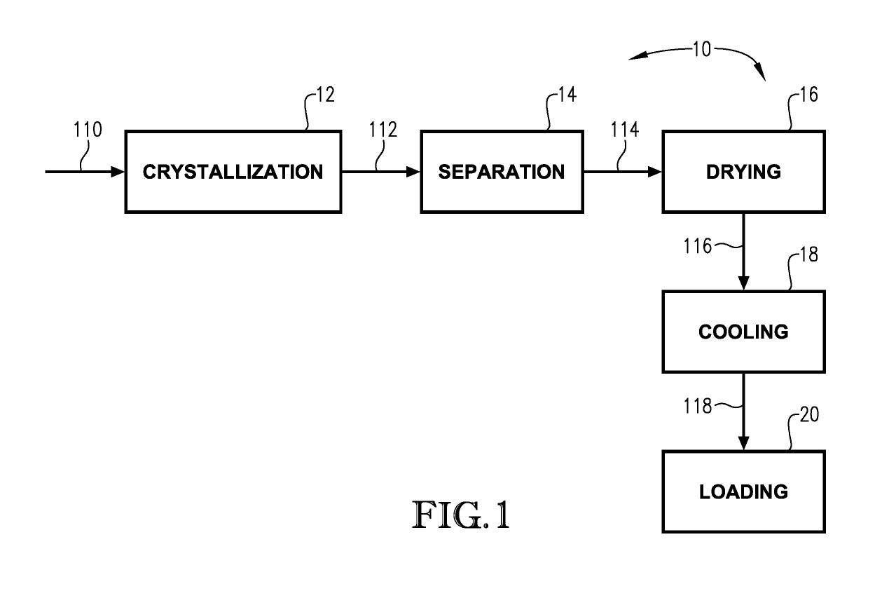

Turning initially to FIG. 1, a schematic depiction of a hydroquinone crystallization and recovery facility 10 configured according to one embodiment of the present invention is provided. As shown in FIG. 1, facility 10 includes a crystallization zone 12, a separation zone 14, a drying zone 16, a cooling zone 18, and a loading zone 20. In operation, a hydroquinone-containing solution can be introduced into facility 10 via conduit 110 and may thereafter be at least partially crystallized in crystallization zone 12. The resulting crystallization slurry in conduit 112, which includes a plurality of hydroquinone crystals dispersed in a crystallization liquid, may be introduced into a separation zone 14, wherein at least a portion of the crystallization liquid may be separated from the particles.

The resulting separated hydroquinone particles can be passed from separation zone 14 to a drying zone 16, as shown by conduit 114, and the particles may then be dried, and optionally washed, prior to being cooled in cooling zone 18. The resulting cooled particles may be transported to a loading zone 20, wherein at least a portion of the crystallized hydroquinone can be loaded into at least one storage container before being transported to one or more locations outside of facility 10, wherein the container may be further transported and/or stored. Facility 10 shown in FIG. 1 may be a small- or pilot-scale facility or can be a commercial-size facility having a capable of loading at least about 500, at least about 1000, at least about 1500, at least about 2000, at least about 2500, or at least about 3000 kg/hour of cooled particles into storage containers in loading zone 20.

The hydroquinone-containing solution introduced into facility 10 via conduit 110 can originate from any suitable source and, in one embodiment, may originate from an upstream portion of facility 10 (not shown) to produce hydroquinone. Many different reaction pathways for obtaining hydroquinone are known and may be used. For example, in the upstream reaction zone (not shown), p-diisopropylbenzene and/or p-diisopropylbenzene monohydroyperoxide may be oxidized with molecular oxygen and the resulting p-diisopropylbenzene dihydroperoxide can be decomposed in the presence of a catalyst to form hydroquinone. Alternatively, phenol can be subjected to catalytic oxidation in the presence of, for example, copper-based catalyst, and the resulting p-benzoquinone can be hydrogenated to provide hydroquinone. Alternatively, the hydroquinone-containing solution in conduit 110 can originate from a reaction zone wherein aniline is oxidized with manganese dioxide or sodium dichromate to form quinone, which can then be reduced to provide hydroquinone. Or, the reaction zone may be configured to alkylate benzene with propylene to form p-diisopropylbenzene that is then oxidized to form dihydroperoxide, which can then be rearranged to provide hydroquinone and acetone. After synthesis, a crude hydroquinone solution withdrawn from the reaction zone may be subjected to one or more purification or separation steps (not shown), such as liquid-liquid or solid-liquid separation, filtration, and/or extraction, in order to provide the hydroquinone-containing solution introduced into recovery facility 10 via conduit 110.

The concentration of hydroquinone in the solution in conduit 110 may be at least about 1, at least about 2, at least about 5, at least about 10, at least about 15, at least about 20, or at least about 25 weight percent and/or not more than about 60, not more than about 50, not more than about 45, not more than about 40, or not more than about 35 weight percent, based on the total weight of the solution, or the concentration can be in the range of from about 1 to about 60 weight percent, about 1 to about 50 weight percent, about 1 to about 45 weight percent, about 1 to about 40 weight percent, about 1 to about 35 weight percent, about 2 to about 60 weight percent, about 2 to about 50 weight percent, about 2 to about 45 weight percent, about 2 to about 40 weight percent, about 2 to about 35 weight percent, about 5 to about 60 weight percent, about 5 to about 50 weight percent, about 5 to about 45 weight percent, about 5 to about 40 weight percent, about 5 to about 45 weight percent, about 10 to about 60 weight percent, about 10 to about 50 weight percent, about 10 to about 45 weight percent, about 10 to about 40 weight percent, about 10 to about 35 weight percent, about 15 to about 60 weight percent, about 15 to about 50 weight percent, about 15 to about 45 weight percent, about 15 to about 40 weight percent, about 15 to about 35 weight percent, about 20 to about 60 weight percent, about 20 to about 50 weight percent, about 20 to about 45 weight percent, about 20 to about 40 weight percent, about 20 to about 35 weight percent, about 25 to about 60 weight percent, about 25 to about 50 weight percent, about 25 to about 45 weight percent, about 25 to about 40 weight percent, or about 25 to about 35 weight percent, based on the total weight of the solution.

The solvent can be any suitable solvent. In one embodiment, the solvent is an aqueous solvent that may, for example, comprise at least about 10, at least about 15, at least about 20, at least about 25, at least about 30, at least about 40, at least about 50, at least about 55, at least about 60, at least about 75, at least about 80, or at least about 95 percent water, or it can be water. The solvent used in the hydroquinone-containing solution in conduit 110 may include at least one organic component, or it may substantially or entirely exclude organic components. Embodiments exist in which the solvent of the solution in conduit 110 can comprise less than about 5, less than about 2, less than about 1, or less than about 0.5 weight percent of one or more organic components, based on the total weight of the solution.

As shown in FIG. 1, the hydroquinone-containing solution introduced into recovery facility 10 can be introduced via conduit 110 into a crystallization zone 12. Crystallization zone 12 can include one or more crystallization vessels or reactors configured to cool and at least partially crystallize the hydroquinone from the solution in conduit 110. Embodiments exist in which crystallization zone 12 can employ at least 1, at least 2, at least 3, at least 4 and/or not more than 10, not more than 8, or not more than 6 separate crystallization vessels, which may be arranged in series or in parallel, and can be operated in a batch, semi-batch, or continuous manner. The number of crystallization vessels employed in crystallization zone 12 can be in the range of from 1 to 10, 1 to 8, 1 to 6, 2 to 10, 2 to 8, 2 to 6, 3 to 10, 3 to 8, 3 to 6, 4 to 10, 4 to 8, 4 to 6. The vessels utilized in crystallization zone 12 may or may not include an agitator or other internal devices.

Crystallization zone 12 may be configured to cool the solution in conduit 110 from an initial temperature, T₁, to a final crystallization temperature, T_{CF}. Each of the temperatures mentioned herein, including, for example, T₁, T₂, T₃, T_{C}, T_{C1}) T_{C2}, T_{C3}, T_{C4}, and T_{CF}, refer to the temperature of the solution or slurry measured inside the surface of the vessel or container, when the solution is agitated, unless otherwise noted. The initial temperature, T₁, can be at least about 65, at least about 70, at least about 75, at least about 80° C. and/or not more than about 100, not more than about 95, not more than about 90, or not more than about 85° C., or a temperature in the range of from about 65 to about 100° C., about 65 to about 95° C., about 65 to about 90° C., about 65 to 85° C., about 70 to about 100° C., about 70 to about 95° C., about 70 to about 90° C., about 70 to about 85° C., about 75 to about 100° C., about 75 to about 95° C., about 75 to about 90° C., about 75 to about 85° C., about 80 to about 100° C., about 80 to about 95° C., about 80 to about 90° C., or about 80 to about 85° C. The final crystallization temperature of the crystallization slurry, T_{CF}, can be at least about 5, at least about 10, at least about 12° C. and/or not more than about 30, not more than about 27° C., not more than about 25, not more than about 20, or not more than about 17° C., or can be in the range of from about 5 to about 30° C., about 5 to about 27° C., about 5 to about 25° C., about 5 to about 20° C., about 5 to about 17° C., about 10 to about 30° C., about 10 to about 27° C., about 10 to about 25° C., about 10 to about 20° C., about 10 to about 17° C., about 12 to about 30° C., about 12 to about 27° C., about 12 to about 25° C., about 12 to about 20° C., or about 12 to about 17° C.

The cooling of the hydroquinone solution and/or slurry performed in crystallization zone 12 can be carried out via evaporative cooling, such that the pressure of crystallization zone 12 can be reduced during all, or a portion, of the crystallization step. Embodiments exist in which the pressure of crystallization zone 12 may be maintained at or below atmospheric pressure. Although the pressure may change, or be reduced, during all or a portion of the crystallizing step, the average value of the pressure of crystallization zone 12, over the entire crystallizing stage, may be at least about 10, at least about 25, at least about 50, at least about 75, at least about 100 millibar (mb) and/or not more than about 500, not more than about 450, not more than about 400, not more than about 350, not more than about 300, or not more than about 250 mb, or in the range of from 10 to about 500 mb, about 10 to about 450 mb, about 10 to about 400 mb, about 10 to about 350 mb, about 10 to about 300 mb, about 10 to about 250 mb, about 25 to about 500 mb, about 25 to about 450 mb, about 25 to about 400 mb, about 25 to about 350 mb, about 25 to about 300 mb, about 25 to about 250 mb, about 50 to about 500 mb, about 50 to about 450 mb, about 50 to about 400 mb, about 50 to about 350 mb, about 50 to about 300 mb, about 50 to about 250 mb, about 75 to about 500 mb, about 75 to about 450 mb, about 75 to about 400 mb, about 75 to about 350 mb, about 75 to about 300 mb, about 75 to about 250 mb, about 100 to about 500 mb, about 100 to about 450 mb, about 100 to about 400 mb, about 100 to about 350 mb, about 100 to about 300 mb, or about 100 to about 250 mb. Embodiments exist in which, as the solution cools, the pressure can be reduced, such that the pressure during an early portion of the cooling and/or crystallization can be higher than the average pressure during a later portion of the cooling and/or crystallization.

The cooling of the hydroquinone solution and/or slurry performed in crystallization zone 12 can be carried out in one or more cooling stages and may optionally include one or more “hold” periods, wherein the temperature and/or pressure of the solution and/or slurry is maintained relatively constant for a period of time. Such hold periods can be carried out before and/or after crystallization of the hydroquinone. For example, embodiments exist in which the hydroquinone-containing solution can be cooled from its initial temperature, T₁, to a crystallization temperature (or “crystallization temperature”), T_{C}, whereupon at least a portion of the dissolved hydroquinone can crystallize out of solution during an initial hold period. After completion of the initial hold period, the cooled hydroquinone-containing solution in crystallization zone 12 may be further cooled from a first crystallization temperature, T_{C1}, to the final crystallization temperature, T_{CF}, via one or more additional cooling periods, optionally with one or more further hold periods.

When the cooling of the hydroquinone-containing solution from T₁ to T_{C} is performed in two or more initial cooling stages, each of the initial cooling stages may have a different cooling rate. For example, the hydroquinone-containing solution in crystallization zone 12 may be cooled from its initial temperature, T₁, to a first intermediate temperature, T₂, to provide a cooled solution and, thereafter, the cooled solution can optionally be further cooled to a second intermediate temperature, T₃. As mentioned previously, temperatures provided herein for the crystallization solution and/or slurry refer to the temperature of the solution or slurry measured inside the surface of the vessel or container, when the solution is agitated.

Embodiments exist in which T₂ can be at least about 65, at least about 66, at least about 67, at least about 68, at least about 69, at least about 70° C. and/or not more than about 77, not more than about 76, not more than about 75, not more than about 74, not more than about 73, not more than about 72, not more than about 71, or not more than about 70° C., or in the range of from about 65 to about 77° C., about 65 to about 76° C., about 65 to about 75° C., about 65 to about 74° C., about 65 to about 73° C., about 65 to about 72° C., about 65 to about 71° C., about 65 to about 70° C., about 66 to about 77° C., about 66 to about 76° C., about 66 to about 75° C., about 66 to about 74° C., about 66 to about 73° C., about 66 to about 72° C., about 66 to about 71° C., about 66 to about 70° C., about 67 to about 77° C., about 67 to about 76° C., about 67 to about 75° C., about 67 to about 74° C., about 67 to about 73° C., about 67 to about 72° C., about 67 to about 71° C., about 67 to about 70° C., about 68 to about 77° C., about 68 to about 76° C., about 68 to about 75° C., about 68 to about 74° C., about 68 to about 73° C., about 68 to about 72° C., about 68 to about 71° C., about 68 to about 70° C., about 69 to about 77° C., about 69 to about 76° C., about 69 to about 75° C., about 69 to about 74° C., about 69 to about 73° C., about 69 to about 72° C., about 69 to about 71° C., about 69 to about 70° C., about 70 to about 77° C., about 70 to about 76° C., about 70 to about 75° C., about 70 to about 74° C., about 70 to about 73° C., about 70 to about 72° C., or about 70 to about 71° C.

When the solution in crystallization zone 12 is cooled to a second intermediate temperature, T₃ can have a value of at least about 64, at least about 64.5, at least about 65, at least about 65.5, at least about 66, at least about 66.5, at least about 67° C. and/or not more than about 72, not more than about 71.5, not more than about 71, not more than about 70.5, not more than about 70, or not more than about 69.5° C., or in the range of from about 64 to about 72° C., about 64 to about 71.5° C., about 64 to about 71° C., about 64 to about 70.5° C., about 64 to about 70° C., about 64 to about 69.5° C., about 64.5 to about 72° C., about 64.5 to about 71.5° C., about 64.5 to about 71° C., about 64.5 to about 70.5° C., about 64.5 to about 70° C., about 64.5 to about 69.5° C., about 65 to about 72° C., about 65 to about 71.5° C., about 65 to about 71° C., about 65 to about 70.5° C., about 65 to about 70° C., about 65 to about 69.5° C., about 65.5 to about 72° C., about 65.5 to about 71.5° C., about 65.5 to about 71° C., about 65.5 to about 70.5° C., about 65.5 to about 70° C., about 65.5 to about 69.5° C., about 66 to about 72° C., about 66 to about 71.5° C., about 66 to about 71° C., about 66 to about 70.5° C., about 66 to about 70° C., about 66 to about 69.5° C., about 66.5 to about 72° C., about 66.5 to about 71.5° C., about 66.5 to about 71° C., about 66.5 to about 70.5° C., about 66.5 to about 70° C., about 66.5 to about 69.5° C., about 67 to about 72° C., about 67 to about 71.5° C., about 67 to about 71° C., about 67 to about 70.5° C., about 67 to about 70° C., or about 67 to about 69.5° C.

The first initial cooling stage, which can cool the solution from T₁ to T₂, can be performed for a time period of at least at least about 2, at least about 5, at least about 10 minutes and/or not more than about 35, not more than about 30, not more than about 25, or not more than about 20, or a time period in the range of from about 2 to about 35 minutes, about 2 to about 30 minutes, about 2 to about 25 minutes, about 2 to about 20 minutes, about 5 to about 35 minutes, about 5 to about 30 minutes, about 5 to about 25 minutes, about 5 to about 20 minutes, about 10 to about 35 minutes, about 10 to about 30 minutes, about 10 to about 25 minutes, or about 10 to about 20 minutes.

The second initial cooling stage, which can cool the solution from T₂ to T₃, or, if no second intermediate temperature is used, from T₂ to T_{C}, can be carried out for a period of at least about 2, at least about 5, at least about 10, at least about 15 minutes and/or not more than about 40, not more than about 35, not more than about 30, or not more than about 25 minutes, or for a time period in the range of from about 2 to about 40 minutes, about 2 to about 35 minutes, about 2 to about 30 minutes, about 2 to about 25 minutes, about 5 to about 40 minutes, about 5 to about 35 minutes, about 5 to about 30 minutes, about 5 to about 25 minutes, about 10 to about 40 minutes, about 10 to about 35 minutes, about 10 to about 30 minutes, about 10 to about 25 minutes, about 15 to about 40 minutes, about 15 to about 35 minutes, about 15 to about 30 minutes, or about 15 to about 25 minutes. The first initial cooling stage can be longer than the second initial cooling stage, approximately the same length as the second initial cooling stage, or shorter than the second initial cooling stage.

The first initial cooling stage can be performed at a first average cooling rate, r_{C1}, while the second initial cooling stage can be performed at a second average cooling rate, r_{C2}. The first and second average cooling rates, r_{C1} and r_{C2}, may be different from one another and, in one embodiment, r_{C2} can be less (slower) than r_{C1}. In general, the average cooling rate between a first temperature, T_{A}, and a second temperature, T_{B}, respectively taken at a first time, t_{A}, and a second time, t_{B}, which is later than t_{A}, can be calculated according to the following formula: (T_{B}−T_{A})/(t_{B}−t_{A}). Thus, the average cooling rate between two points, T_{B} and T_{A}, is equal to the slope of a straight line drawn between the two points, as plotted on a temperature versus time graph. Accordingly, it should be understood that the instantaneous rate of cooling of the crystallization solution at any given time between t_{A} and t_{B} may vary slightly, or even significantly, but the solution may still have an average cooling rate between T_{A} and T_{B} as defined above.

Embodiments exist in which r_{C1} can be at least about 0.010, at least about 0.050, at least about 0.10, at least about 0.25, at least about 0.50 and/or not more than about 10, not more than about 5, or not more than about 2° C./min, or it may be in the range of from about 0.010 to about 10° C./min, about 0.010 to about 5° C./min, about 0.010 to about 2° C./min, about 0.50 to about 10° C./min, about 0.50 to about 5° C./min, about 0.50 to about 2° C./min, about 0.10 to about 10° C./min, about 0.10 to about 5° C./min, about 0.10 to about 2° C./min, about 0.25 to about 10° C./min, about 0.25 to about 5° C./min, about 0.25 to about 2° C./min, about 0.50 to about 10° C./min, about 0.50 to about 5° C./min, or about 0.50 to about 2° C./min.

The second average cooling rate, r_{C2}, can be at least about 0.025, at least about 0.050, at least about 0.1, at least about 0.25, at least about 0.50, and/or not more than about 10, not more than about 8, not more than about 5, not more than about 4, not more than about 2, not more than about 1, or not more than about 0.075° C./min, or can be in the range of from about 0.025 to about 10° C./min, about 0.025 to about 8° C./min, about 0.025 to about 5° C./min, about 0.025 to about 4° C./min, about 0.025 to about 2° C./min, about 0.025 to about 1° C./min, about 0.025 to about 0.75° C./min, about 0.050 to about 10° C./min, about 0.050 to about 8° C./min, about 0.050 to about 5° C./min, about 0.050 to about 4° C./min, about 0.050 to about 2° C./min, about 0.050 to about 1° C./min, about 0.050 to about 0.75° C./min, about 0.25 to about 10° C./min, about 0.25 to about 8° C./min, about 0.25 to about 5° C./min, about 0.25 to about 4° C./min, about 0.25 to about 2° C./min, about 0.25 to about 1° C./min, about 0.25 to about 0.75° C./min, about 0.5 to about 10° C./min, about 0.5 to about 8° C./min, about 0.5 to about 5° C./min, about 0.5 to about 4° C./min, about 0.5 to about 2° C./min, about 0.5 to about 1° C./min, or about 0.5 to about 0.75° C./min.

The ratio of the first average cooling rate to the second average cooling rate (r_{C1}:r_{C2}) can be at least about 2:1, at least about 3:1, at least about 4:1, at least about 5:1, at least about 6:1, at least about 8:1, at least about 10:1 and/or not more than about 25:1, not more than about 20:1, not more than about 18:1, or not more than about 15:1, or in the range of from about 2:1 to about 25:1, about 2:1 to about 20:1, about 2:1 to about 18:1, about 2:1 to about 15:1, about 3:1 to about 25:1, about 3:1 to about 20:1, about 3:1 to about 18:1, about 3:1 to about 15:1, about 4:1 to about 25:1, about 4:1 to about 20:1, about 4:1 to about 18:1, about 4:1 to about 15:1, about 5:1 to about 25:1, about 5:1 to about 20:1, about 5:1 to about 18:1, about 5:1 to about 15:1, about 6:1 to about 25:1, about 6:1 to about 20:1, about 6:1 to about 18:1, about 6:1 to about 15:1, about 8:1 to about 25:1, about 8:1 to about 20:1, about 8:1 to about 18:1, about 8:1 to about 15:1, about 10:1 to about 25:1, about 10:1 to about 20:1, about 10:1 to about 18:1, or about 10:1 to about 15:1.

Additionally, one or more additional initial cooling stages can also be employed in order to reduce the temperature of the solution even further to a temperature at or near the onset of crystallization. For example, a third intermediate cooling stage can also be utilized in crystallization zone 12 to additionally cool the further cooled solution from the second intermediate temperature, T₃, to a third intermediate temperature, T₄. Embodiments exist in which T₃ can be at least about 65, at least about 65.25, at least about 65.5, at least about 65.75, at least about 66, at least about 66.25° C. and/or not more than about 69, not more than about 68.5, not more than about 68, not more than about 67.5, not more than about 67, or not more than about 66.75° C., or in the range of from about 65 to about 69° C., about 65 to about 68.5° C., about 65 to about 68° C., about 65 to about 67.5° C., about 65 to about 67° C., about 65 to about 66.75° C., about 65.25 to about 69° C., about 65.25 to about 68.5° C., about 65.25 to about 68° C., about 65.25 to about 67.5° C., about 65.25 to about 67° C., about 65.25 to about 66.75° C., about 65.5 to about 69° C., about 65.5 to about 68.5° C., about 65.5 to about 68° C., about 65.5 to about 67.5° C., about 65.5 to about 67° C., about 65.5 to about 66.75° C., about 65.75 to about 69° C., about 65.75 to about 68.5° C., about 65.75 to about 68° C., about 65.75 to about 67.5° C., about 65.75 to about 67° C., about 65.75 to about 66.75° C., about 66 to about 69° C., about 66 to about 68.5° C., about 66 to about 68° C., about 66 to about 67.5° C., about 66 to about 67° C., about 66 to about 66.75° C., about 66.25 to about 69° C., about 66.25 to about 68.5° C., about 66.25 to about 68° C., about 66.25 to about 67.5° C., about 66.25 to about 67° C., or about 66.25 to about 66.75° C.

When employed, the third intermediate temperature, T₄ can be within about 0.1, within about 0.5, within about 1, within about 2, within about 2.5, within about 5° C. of the crystallization temperature, T_{C}. At least a portion of the cooling performed in any of the initial cooling stages may be carried out using evaporative cooling. In addition, or in the alternative, at least a portion of the cooling of the solution from T₁ to T_{C} may be carried out via indirect heat exchange with one or more heat transfer fluids, such as, for example, cooling water or other cooling fluid passed through a jacket or internal exchanger disposed in the crystallization or cooling vessel.

When present, the average cooling rate of the third initial cooling stage, r_{C3}, can be less than (slower than) the cooling rate of the second initial cooling stage, r_{C2}. Embodiments exist in which r_{C3} can be at least about 0.01, at least about 0.05, at least about 0.1, at least about 0.5, at least about 0.75° C./min and/or not more than about 10, not more than about 8, not more than about 6, not more than about 5, not more than about 4, not more than about 2, or not more than about 1° C./min, or in the range of from about 0.01 to about 10° C./min, about 0.01 to about 8° C./min, about 0.01 to about 6° C./min, about 0.01 to about 5° C./min, about 0.01 to about 4° C./min, about 0.01 to about 2° C./min, about 0.01 to about 1° C./min, about 0.05 to about 10° C./min, about 0.05 to about 8° C./min, about 0.05 to about 6° C./min, about 0.05 to about 5° C./min, about 0.05 to about 4° C./min, about 0.05 to about 2° C./min, about 0.05 to about 1° C./min, about 0.1 to about 10° C./min, about 0.1 to about 8° C./min, about 0.1 to about 6° C./min, about 0.1 to about 5° C./min, about 0.1 to about 4° C./min, about 0.1 to about 2° C./min, about 0.1 to about 1° C./min, about 0.5 to about 10° C./min, about 0.5 to about 8° C./min, about 0.5 to about 6° C./min, about 0.5 to about 5° C./min, about 0.5 to about 4° C./min, about 0.5 to about 2° C./min, about 0.5 to about 1° C./min, about 0.75 to about 10° C./min, about 0.75 to about 8° C./min, about 0.75 to about 6° C./min, about 0.75 to about 5° C./min, about 0.75 to about 4° C./min, about 0.75 to about 2° C./min, or about 0.75 to about 1° C./min.

The ratio of the second average cooling rate, r_{C2}, to the third average cooling rate, r_{C3}, can be at least about 2:1, at least about 3:1, at least about 4:1, at least about 5:1, at least about 6:1, at least about 8:1, at least about 10:1 and/or not more than about 25:1, not more than about 20:1, not more than about 18:1, not more than about 15:1, or not more than about 13:1, or the ratio may be in the range of from about 2:1 to about 25:1, about 2:1 to about 20:1, about 2:1 to about 18:1, about 2:1 to about 15:1, about 2:1 to about 13:1, about 3:1 to about 25:1, about 3:1 to about 20:1, about 3:1 to about 18:1, about 3:1 to about 15:1, about 3:1 to about 13:1, about 4:1 to about 25:1, about 4:1 to about 20:1, about 4:1 to about 18:1, about 4:1 to about 15:1, about 4:1 to about 13:1, about 5:1 to about 25:1, about 5:1 to about 20:1, about 5:1 to about 18:1, about 5:1 to about 15:1, about 5:1 to about 13:1, about 6:1 to about 25:1, about 6:1 to about 20:1, about 6:1 to about 18:1, about 6:1 to about 15:1, about 6:1 to about 13:1, about 8:1 to about 25:1, about 8:1 to about 20:1, about 8:1 to about 18:1, about 8:1 to about 15:1, about 8:1 to about 13:1, about 10:1 to about 25:1, about 10:1 to about 20:1, about 10:1 to about 18:1, about 10:1 to about 15:1, or about 10:1 to about 13:1.

Additionally, r_{C3} may also be less than (slower than) the average cooling rate utilized during the first initial cooling stage, r_{C1}. The ratio of r_{C1} to r_{C3} (r_{C1}:r_{C3}) can be at least about 10:1, at least about 25:1, at least about 50:1 and/or not more than about 250:1, not more than about 200:1, not more than about 150:1, not more than about 100:1, or not more than about 75:1, or in the range of from about 10:1 to about 250:1, about 10:1 to about 200:1, about 10:1 to about 150:1, about 10:1 to about 100:1, about 10:1 to about 75:1, about 25:1 to about 250:1, about 25:1 to about 200:1, about 25:1 to about 150:1, about 25:1 to about 100:1, about 25:1 to about 75:1, about 50:1 to about 250:1, about 50:1 to about 200:1, about 50:1 to about 150:1, about 50:1 to about 100:1, or about 50:1 to about 75:1. Any number of additional cooling stages may also be utilized to cool the hydroquinone-containing solution from T₁ to T_{C}, and, when present, each of the additional cooling stages can also include cooling rates lower (slower) than one or more of the cooling rates of the preceding cooling stages. Preferably, each of the cooling stages and the subsequent crystallization can be performed in the same vessel, although one or more stages may be performed in separate vessels.

Embodiments exist in which the crystallization solution in crystallization zone 12 may be seeded during one or more of the above-described cooling stages. For example, a plurality of seed crystals may be added to the solution in crystallization zone 12 once the solution has been cooled to the first intermediate temperature, T₂, as discussed above. The form of the seed crystals is not particularly limited, and the crystals may be added into crystallization zone 12 in the form of a solution, as a dried powder, or combinations thereof. Embodiments exist in which the median particle size of the seed crystals can be less than about 125 microns, less than about 100 microns, or less than 75 microns. The amount of seed crystals can also vary, but embodiments exist in which the seed crystals may be added to the crystallization solution in an amount that can be at least about 0.5, at least about 1, at least about 2 weight percent and/or not more than about 8, not more than about 6, or not more than about 5 weight percent, based on the weight of the crystallization solution in zone 12 prior to addition of the seed crystals, or the amount can be in the range of from about 0.5 to about 8 weight percent, about 0.5 to about 6 weight percent, about 0.5 to about 5 weight percent, about 1 to about 8 weight percent, about 1 to about 6 weight percent, about 1 to about 5 weight percent, about 2 to about 8 weight percent, about 2 to about 6 weight percent, or about 2 to about 5 weight percent.

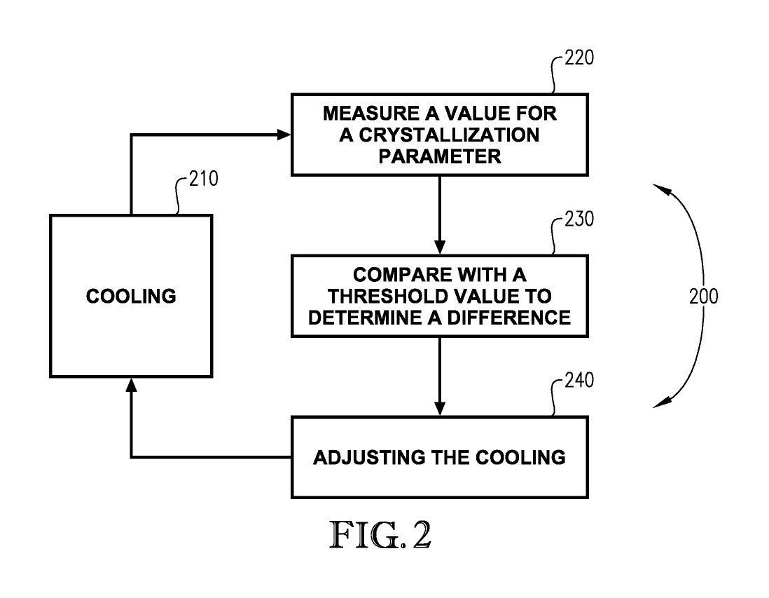

Turning now to FIG. 2, a flowchart representing certain steps of a method 200 for determining the onset of crystallization of the hydroquinone-containing solution during at least a portion of the cooling carried out in crystallization zone 12 is provided. As shown in FIG. 2, the steps of method 200 may be performed during at least a portion of the cooling of the hydroquinone-containing solution, which is represented by block 210. The onset of crystallization may be the point at which crystallization of the hydroquinone begins, and may occur, for example, when the first crystal is formed up to when less than about 20, less than about 15, less than about 10, less than about 5, or less than about 2 percent of the total amount of hydroquinone in solution has been crystallized. Upon identification of the onset of crystallization for the solution in crystallization zone 12, the cooling of the solution may be stopped and crystallization zone 12 held at substantially constant temperature and/or pressure in order for the remainder of the hydroquinone to crystallize out of solution. All or a portion of method 200 shown in FIG. 2 may be carried out with an automated control system.

As shown in FIG. 2, method 200 can include the step of measuring the value of at least one crystallization parameter in the crystallization zone in order to provide a measured value, as represented by block 220. As used herein, the term “crystallization parameter,” refers to a parameter used to determine the presence or extent of crystallization. Examples of suitable crystallization parameters that can be measured include indirect crystallization parameters and direct crystallization parameters. As used herein, the term “indirect crystallization parameter,” refers to a crystallization parameter that is not the result of direct observation of the crystallized particles or solution, but that is indicative of some aspect of crystallization. Examples of indirect crystallization parameters can include, but are not limited to, temperature or total elapsed cooling time. As used herein, the term “direct crystallization parameter” refers to a crystallization parameter that is measured by directly observing one or more characteristics of the crystallized particles or solution, including, for example, parameters related to the type, size, or number of particles. Examples of suitable direct crystallization parameters can include, but are not limited to, total particle count, normalized particle count, particle development rate, average particle size, median particle size, particle size distribution, and chord length distribution. Embodiments exist in which direct crystallization parameters related to the size of the particles, such as average particle size, median particle size, and particle size distribution, may not be measured.

When the parameter measured during the step of method 200 represented by block 220 includes a direct crystallization parameter, all or a portion of the measuring step may be carried out by visual observation of at least a portion of the hydroquinone-containing solution in crystallization zone 12. Visual observation, when used, can be performed by a human observer and/or may be carried out automatically with visual imaging equipment. When all or a portion of the visual observation is performed with visual imaging equipment, that may include, for example a camera, the visual observation may be taken over all or a portion of the camera's total view area. As used herein, the term “total view area” refers to the total area of view of a camera and is calculated by multiplying the horizontal and vertical fields of view of that camera as it is used to monitor the crystallization zone. Each of the horizontal and vertical fields of view are calculated based on the type of camera lens used, the working distance between the lens and a designated point within the crystallization zone (such as, for example, a point on the arm of the agitator in the middle of the crystallizer), and the specific camera sensor. Horizontal and vertical fields of view are defined by Equations (1) and (2), below.

$$\begin{matrix}\frac{Sensor Size ( Horizontal ) * Working Distance}{Focal Length} = Horizontal Field of View & ( 1 ) \\ \frac{Sensor Size ( Vertical ) * Working Distance}{Focal Length} = Vertical Field of View & ( 2 )\end{matrix}$$

Embodiments exist in which the total view area can be at least about 1500, at least about 2500, at least about 3500 cm² and/or not more than about 10,000, not more than about 7500, or not more than about 5500 cm², or the total view area can be in the range of from about 1500 to about 10,000 cm², about 1500 to about 7500 cm², about 1500 to about 5500 cm², about 2500 to about 10,000 cm², about 2500 to about 7500 cm², about 2500 to about 5500 cm², about 3500 to about 10,000 cm², about 3500 to about 7500 cm², or about 3500 to about 5500 cm². The visual observation can be performed over the entirety of the total view area, or over a smaller measurement area defined over a portion of the total view area.

As used herein, the term “measurement area” refers to the portion of the total view area over which a visual measurement or observation is formed. Visual observation can be performed over a measurement area that has an area smaller than the total view area. For example, embodiments exist in which the area of the measurement area is at least about 10, at least about 20, at least about 30 percent of the total view area and/or not more than about 70 percent, not more than about 60 percent, or not more than about 50 percent of the total view area, or the measurement area can have an area that is in the range of from about 10 to about 70 percent, about 10 to about 60 percent, about 10 to about 50 percent, about 20 to about 70 percent, about 20 to about 60 percent, about 20 to about 50 percent, about 30 to about 70 percent, about 30 to about 60 percent, or about 30 to about 50 percent of the total view area.

Embodiments exist in which the measurement area can be at least about 10, at least about 50, at least about 100, at least about 500, at least about 1000 cm² and/or not more than about 2500, not more than about 2000, or not more than about 1750 cm², or can be in the range of from about 10 to about 2500 cm², about 10 to about 2000 cm², about 10 to about 1750 cm², about 50 to about 2500 cm², about 50 to about 2000 cm², about 50 to about 1750, about 100 to about 2500 cm², about 100 to about 2000 cm², about 100 to about 1750 cm², about 500 to about 2500 cm², about 500 to about 2000 cm², about 500 to about 1750, about 1000 to about 2500 cm², about 1000 to about 2000 cm², or about 1000 to about 1750 cm². Embodiments exist in which the measurement area can be smaller, such as, for example, less than 10 cm², less than 5 cm², or less than 2 cm².

As used herein, the term “total particle count” refers to the total number of particles observed within a specified measurement area, while the term “normalized particle count,” refers to the number of particles observed within a specified measurement area, normalized by the total area of the measurement area. For example, in a measurement area having an area of 10 cm² and including 100 particles, the total particle count for that measurement area would be 100, while the normalized particle count would be 100 particles÷ 10 cm², or 10 particles/cm². The total particle count can be measured as an instantaneous or near instantaneous value, or may be calculated as a moving average. The term “particle development rate,” as used herein refers to the number of particles formed in a specified unit of time. The particle development rate can be determined by dividing the total number of particles formed during a period of time by the amount of time, or, instantaneously, by calculating the slope of a graph of the total particle count versus time.

Once the value of the selected crystallization parameter or parameters has been measured, the measured value can then be compared with a predetermined threshold value for that crystallization parameter in order to determine a difference, a step that is represented by block 230 in FIG. 2.

When the crystallization parameter includes total particle count, the threshold value can be at least about 15, at least about 20, at least about 30 particles and/or not more than about 200, not more than about 100, or not more than about 75 particles, or it can be in the range of from about 15 to about 200 particles, about 15 to about 100 particles, about 15 to about 75 particles, about 20 to about 200 particles, about 20 to about 100 particles, about 20 to about 75 particles, about 30 to about 200 particles, about 30 to about 100 particles, or about 30 to about 75 particles. When the selected crystallization parameter includes normalized particle count, the threshold value can be at least about 50, at least about 100, at least about 200 particles/m² and/or not more than about 1200, not more than about 1000, or not more than about 750 particles/m², or it can be in the range of from about 50 to about 1200 particles/m², about 50 to about 1000 particles/m², about 50 to about 750 particles/m², about 100 to about 1200 particles/m², about 100 to about 1000 particles/m², about 100 to about 750 particles/m², about 200 to about 1200 particles/m², about 200 to about 1000 particles/m², or about 200 to about 750 particles/m².

When the crystallization parameter includes particle development rate, the threshold value used to compare with the measured value can be at least about 0.5, at least about 1, at least about 1.5, at least about 2 particles/min and/or not more than about 10, not more than about 8, not more than about 6, or not more than about 4 particles/min, or it can be in the range of from about 0.5 to about 10 particles/min, about 0.5 to about 8 particles/min, about 0.5 to about 6 particles/min, about 0.5 to about 4 particles/min, about 1 to about 10 particles/min, about 1 to about 8 particles/min, about 1 to about 6 particles/min, about 1 to about 4 particles/min, about 1.5 to about 10 particles/min, about 1.5 to about 8 particles/min, about 1.5 to about 6 particles/min, about 1.5 to about 4 particles/min, about 2 to about 10 particles/min, about 2 to about 8 particles/min, about 2 to about 6 particles/min, or about 2 to about 4 particles/min.

Comparison of the measured value to the threshold value can be done according to any suitable method, and embodiments exist in which it can be performed with an automated control system. When the crystallization parameter is a direct crystallization parameter, one or more automatic visual detection devices and/or comparison devices may be used to determine the difference between the measured value and the predetermined threshold value. For example, one or more cameras configured to image all or a portion of the crystallization solution within crystallization zone 12 shown in FIG. 1, may be used to capture digital images of the crystallization solution, which may then be transmitted to and/or analyzed by a digital imaging software, such as, for example, LABVIEW software (commercially available from National Instruments in Austin, Tex., USA). Examples of suitable cameras can include, for example, progressive scan CCD cameras, including, for example, those the Basler Ace acA1300-30 gm Monochrome GigE Camera (commercially available from Basler AG in Ahrensburg, Germany). Embodiments exist in which the data collection and analysis performed by the digital imaging equipment can be performed and output provided at or near real-time. The digital imaging equipment can be configured to operate within the automated control system used to control the operation of the crystallization zone or may be configured with its own separate automation system.

As shown in FIG. 2, once a difference has been determined, the cooling performed in crystallization zone 12 may be adjusted, based on the difference, to thereby cause at least a portion of the hydroquinone to crystallize out of solution, as represented by block 240 in FIG. 2. Embodiments exist in which the adjusting step represented by block 240 in FIG. 2 is not performed until the difference between the measured value and the threshold value is at least about 2, at least about 5, at least about 10, at least about 15 percent and/or not more than about 50, not more than about 30, or not more than about 25 percent of the total threshold value. The adjusting step may be performed when the difference is in the range of from about 2 to about 50 percent, about 2 to about 30 percent, about 2 to about 25 percent, about 5 to about 50 percent, about 5 to about 30 percent, about 5 to about 25 percent, about 10 to about 50 percent, about 10 to about 30 percent, about 10 to about 25 percent, about 15 to about 50 percent, about 15 to about 30 percent, or about 15 to about 25 percent of the total threshold value. The step of adjusting the cooling represented by block 240 can include, for example, reducing the cooling rate, increasing the cooling rate, stopping the cooling, or combinations thereof. As discussed previously, the cooling of the hydroquinone-containing solution can be carried out via evaporative cooling of the solvent and/or via indirect contact with at least one cooling medium.

Once the temperature of the solution has been cooled to the crystallization temperature, T_{C}, the temperature and/or pressure of the solution within crystallization zone 12 may be held substantially constant during an initial hold period. As used herein, the term “substantially constant” used with reference to temperature refers to a temperature within about 2° C. of a specified temperature. Embodiments exist in which the temperature of the solution within crystallization zone 12 is held at a temperature within about 5° C., within about 3° C., within about 1° C., within about 0.5° C., or within about 0.25° C. of a specified temperature during the initial hold period. The cooling rate during the initial hold period can be less than 0.0001, less than 0.00005, or less than 0.00001° C./s, as measured between the crystallization temperature, T_{C}, and the temperature of the solution at the end of the initial hold period, first crystallization temperature, T_{C1}. The specified temperature near which the temperature of the solution is maintained during the initial hold period may be the crystallization temperature, T_{C}, or it may be the first crystallization temperature, T_{C1}.

Embodiments exist in which, during the initial hold period, the temperature of the crystallization zone may be maintained within about 5° C., within about 3° C., within about 2° C., within about 1° C., within about 0.5° C., or within about 0.25° C. of at a temperature that is at least about 55, at least about 60, at least about 62, at least about 65° C. and/or not more than about 75, not more than about 70, or not more than about 67° C., or in the range of from about 55 to about 75° C., about 55 to about 70° C., about 55 to about 67° C., about 60 to about 75° C., about 60 to about 70° C., about 60 to about 67° C., about 62 to about 75° C., about 62 to about 70° C., about 62 to about 67° C., about 65 to about 75° C., about 65 to about 70° C., or about 65 to about 67° C. The crystallization temperature, T_{C}, may at least about 50, at least about 55, at least about 60, at least about 62, at least about 65, at least about 70° C., and/or not more than about 80, not more than about 75, not more than about 70, not more than about 67° C., or not more than about 65° C. The crystallization temperature, T_{C}, can be in the range of from about 50 to about 80° C., about 50 to about 75° C., about 50 to about 70° C., about 50 to about 67° C., about 50 to about 65° C., about 55 to about 80° C., about 55 to about 75° C., about 55 to about 70° C., about 55 to about 67° C., about 55 to about 65° C., about 60 to about 75° C., about 60 to about 80° C., about 60 to about 70° C., about 60 to about 67° C., about 60 to 65° C., about 62 to about 75° C., about 62 to about 70° C., about 62 to about 67° C., about 62° C. to about 65° C., about 65 to about 75° C., about 65 to about 70° C., or about 65 to about 67° C.

Alternatively, or in addition, the pressure of crystallization zone 12 may be held substantially constant during the initial hold period. When used with respect to pressure, the phrase “substantially constant,” as used herein, refers to a pressure that is within 10 percent of a specified pressure. Embodiments exist in which the pressure of crystallization zone 12 is maintained at a pressure within about 10 percent of a pressure that is at least about 100, at least about 125, at least about 150, at least about 200 mb, at least about 250 mb and/or not more than about 400 mb, not more than about 350 mb, not more than about 325, or not more than about 250, or at a pressure in the range of from about 100 to about 400 mb, about 100 to about 350 mb, about 100 to about 325 mb, about 100 to about 250, about 125 to about 400 mb, about 125 to about 350 mb, about 125 to about 325 mb, about 125 to about 250 mb, about 150 to about 400 mb, about 150 to about 350 mb, about 150 to about 325, about 150 to about 250 mb, about 200 to about 400 mb, about 200 to about 350 mb, about 200 to about 325, about 200 to about 250 mb, about 250 to about 400 mb, about 250 to about 350 mb, or about 250 to about 325 mb during the initial hold period.

Embodiments exist in which the length of the initial hold period is at least about 5, at least about 10, at least about 15, at least about 20, at least about 25 minutes and/or not more than about 90, not more than about 75, not more than about 60, or not more than about 45 minutes. The length of the initial hold period can be in the range of from about 5 to about 90 minutes, about 5 to about 75 minutes, about 5 to about 60 minutes, about 5 to about 45 minutes, about 10 to about 90 minutes, about 10 to about 75 minutes, about 10 to about 60 minutes, about 10 to about 45 minutes, about 15 to about 90 minutes, about 15 to about 75 minutes, about 15 to about 60 minutes, about 15 to about 45 minutes, about 20 to about 90 minutes, about 20 to about 75 minutes, about 20 to about 60 minutes, about 20 to about 45 minutes, about 25 to about 90 minutes, about 25 to about 75 minutes, about 25 to about 60 minutes, or about 25 to about 45 minutes.

Upon completion of the initial hold period, the crystallization slurry, which may include particles of crystallized hydroquinone dispersed in the crystallization liquid, may be further cooled from a the first crystallization temperature, T_{C1}, to a final temperature, T_{CF}. The first crystallization temperature, T_{C1}, may be within 5, within 3, within 2, within 1, within 0.5, or within 0.25° C. of the crystallization temperature, T_{C}, and can be at least about 55, at least about 60, at least about 65, at least about 65.5, at least about 66, at least about 66.5° C. and/or not more than about 85, not more than about 80, not more than about 75, not more than about 69, not more than about 68, not more than about 67.5, or not more than about 67° C. The first crystallization temperature, T_{C1}, can be in the range of from about 55 to about 85° C., about 55 to about 80° C., about 55 to about 75° C., about 55 to about 69° C., about 55 to about 68° C., about 55 to about 67.5° C., about 55 to about 67° C., about 60 to about 85° C., about 60 to about 80° C., about 60 to about 75° C., about 60 to about 69° C., about 60 to about 68° C., about 60 to about 67.5° C., about 60 to about 67° C., about 65 to about 85° C., about 65 to about 80° C., about 65 to about 75° C., about 65 to about 69° C., about 65 to about 68° C., about 65 to about 67.5° C., about 65 to about 67° C., about 65.5 to about 85° C., about 65.5 to about 80° C., about 65.5 to about 75° C., about 65.5 to about 69° C., about 65.5 to about 68° C., about 65.5 to about 67.5° C., about 65.5 to about 67° C., about 66 to about 85° C., about 66 to about 80° C., about 66 to about 75° C., about 66 to about 69° C., about 66 to about 68° C., about 66 to about 67.5° C., about 66 to about 67° C., about 66.5 to about 85° C., about 66.5 to about 80° C., about 66.5 to about 75° C., about 66.5 to about 69° C., about 66.5 to about 68° C., about 66.5 to about 67.5° C., or about 66.5 to about 67° C. The final crystallization temperature, T_{CF}, can be within one or more of the ranges described previously.

The step of further cooling the crystallized hydroquinone slurry from the first crystallization temperature, T_{C1}, to the final crystallization temperature, T_{CF}, may be performed for a total period of time, Δt, of at least about 15, at least about 30 minutes, at least about 1 hour, at least about 1.5, at least about 2, at least about 2.5, at least about 3, at least about 3.5 hours and/or not more than about 12, not more than about 10, not more than about 8, or not more than about 6 hours, or a period of time in the range of from about 15 minutes to about 12 hours, about 15 minutes to about 10 hours, about 15 minutes to about 8 hours, about 15 minutes to about 6 hours, about 30 minutes to about 12 hours, about 30 minutes to about 10 hours, about 30 minutes to about 8 hours, about 30 minutes to about 6 hours, about 1 hour to about 12 hours, about 1 hour to about 10 hours, about 1 hour to about 8 hours, about 1 hour to about 6 hours, about 1.5 to about 12 hours, about 1.5 to about 10 hours, about 1.5 to about 8 hours, about 1.5 to about 6 hours, about 2 to about 12 hours, about 2 to about 10 hours, about 2 to about 8 hours, about 2 to about 6 hours, about 2.5 to about 12 hours, about 2.5 to about 10 hours, about 2.5 to about 8 hours, about 2.5 to about 6 hours, about 3 to about 12 hours, about 3 to about 10 hours, about 3 to about 8 hours, about 3 to about 6 hours, about 3.5 to about 12 hours, about 3.5 to about 10 hours, about 3.5 to about 8 hours, or about 3.5 to about 6 hours. The step of further cooling the hydroquinone-containing slurry may be carried out in the same vessel as the pre-crystallization cooling and crystallization steps described above, or it may be carried out in one or more separate vessels.

Embodiments exist in which the further cooling of the crystallization slurry from T_{C1} to T_{CF} may be carried out in two or more cooling periods. For example, embodiments exist in which the crystallization slurry in crystallization zone 12 can be cooled from a first crystallization temperature, T_{C1}, to a second crystallization temperature, T_{C2}, during a first crystallization cooling period and, thereafter, the crystallization slurry may then be further cooled from a third crystallization temperature, T_{C3}, to the final crystallization temperature, T_{CF}, during a second crystallization cooling period.

Embodiments exist in which T_{C2} can be less than about 65, less than about 64, less than about 63, less than about 62, less than about 61, less than about 60, less than about 59, less than about 58, less than about 57, less than about 56, or less than about 55° C. After cooling from the first crystallization temperature, T_{C1}, the cooled slurry can have a temperature, T_{C2}, of at least about 35, at least about 40, at least about 45, at least about 47, at least about 50° C. and/or not more than about 60, not more than about 57, not more than about 55° C., or it can be in the range of from about 35 to about 60° C., about 35 to about 57° C., about 35 to about 55° C., about 40 to about 60° C., about 40 to about 57° C., about 40 to about 55° C., about 45 to about 60° C., about 45 to about 57° C., about 45 to about 55° C., about 47 to about 60° C., about 47 to about 57° C., about 47 to about 55° C., about 50 to about 60° C., about 50 to about 57° C., or about 50 to about 55° C.

Embodiments exist in which T_{C3} can be at least about 30, at least about 35, at least about 40, at least about 45° C. and/or not more than about 60, not more than about 57, not more than about 55, not more than about 50° C., or it can be in the range of from about 30 to about 60° C., about 30 to about 57° C., about 30 to about 55° C., about 30 to about 50° C., about 35 to about 60° C., about 35 to about 57° C., about 35 to about 55° C., about 35 to about 50° C., about 40 to about 60° C., about 40 to about 57° C., about 40 to about 55° C., about 40 to about 50° C., about 45 to about 60° C., about 45 to about 57° C., about 45 to about 55° C., or about 45 to about 50° C.

The first crystallization cooling period during which the solution can be cooled from T_{C1} to T_{C2} may have a total length of at least 15, at least about 30 minutes, at least about 1 hour, at least about 1.5 hours, at least about 2 hours and/or not more than 10 hours, not more than about 8 hours, not more than about 6 hours, or not more than about 4 hours. The first crystallization cooling period can have a total duration in the range of from about 15 minutes to about 10 hours, about 15 minutes to about 8 hours, about 15 minutes to about 6 hours, about 15 minutes to about 4 hours, from about 30 minutes to about 10 hours, about 30 minutes to about 8 hours, about 30 minutes to about 6 hours, about 30 minutes to about 4 hours, from about 1 hour to about 10 hours, about 1 hour to about 8 hours, about 1 hour to about 6 hours, about 1 hour to about 4 hours, from about 1.5 hours to about 10 hours, about 1.5 hours to about 8 hours, about 1.5 hours to about 6 hours, about 1.5 hours to about 4 hours, from about 2 hours to about 10 hours, about 2 hours to about 8 hours, about 2 hours to about 6 hours, or about 2 hours to about 4 hours.

Embodiments exist in which the ratio of the length of time of the first crystallization cooling period to the length of time of the initial hold period, which is performed prior to the first crystallization cooling period as discussed previously, can be at least about 0.10:1, at least about 0.25:1, at least about 0.50:1, at least about 0.75:1 and/or not more than about 5:1, not more than about 2:1, not more than about 1.5:1, not more than about 1:1, or the ratio can be in the range of from about 0.10:1 to about 5:1, about 0.10:1 to about 2:1, about 0.10:1 to about 1.5:1, about 0.10:1 to 1:1, about 0.25:1 to about 5:1, about 0.25:1 to about 2:1, about 0.25:1 to about 1.5:1, about 0.25:1 to 1:1, about 0.50:1 to about 5:1, about 0.50:1 to about 2:1, about 0.50:1 to about 1.5:1, about 0.50:1 to 1:1, about 0.75:1 to about 5:1, about 0.75:1 to about 2:1, about 0.75:1 to about 1.5:1, or about 0.75:1 to 1:1.

The second crystallization cooling period during which the solution can be cooled from T_{C3} to T_{CF} may have a total length of at least about 30 minutes, at least about 1 hour, at least about 1.5 hours, at least about 2 hours, at least about 2.5 hours, at least about 3 hours and/or not more than 10 hours, not more than about 8 hours, not more than about 6 hours, or not more than about 4 hours. The second crystallization cooling period can have a total length in the range of from about 30 minutes to about 10 hours, about 30 minutes to about 8 hours, about 30 minutes to about 6 hours, about 30 minutes to about 4 hours, from about 1 hour to about 10 hours, about 1 hour to about 8 hours, about 1 hour to about 6 hours, about 1 hour to about 4 hours, about 1.5 hours to about 10 hours, about 1.5 hours to about 8 hours, about 1.5 hours to about 6 hours, about 1.5 hours to about 4 hours, about 2 hours to about 10 hours, about 2 hours to about 8 hours, about 2 hours to about 6 hours, about 2 hours to about 4 hours, about 2.5 hours to about 10 hours, about 2.5 hours to about 8 hours, about 2.5 hours to about 6 hours, about 2.5 hours to about 4 hours, about 3 hours to about 10 hours, about 3 hours to about 8 hours, about 3 hours to about 6 hours, or about 3 hours to about 4 hours. Any additional crystallization cooling periods, when present, may be performed for a length of time within one or more of the above ranges.

When two or more crystallization cooling periods are utilized during cooling of the crystallization slurry, the crystallization cooling periods may be performed for similar lengths of time, or one or more crystallization cooling periods may be longer than one or more of the others. For example, embodiments exist in which the ratio of the length of time of the first crystallization cooling period to the second crystallization cooling period is at least about 0.5:1, at least about 0.75:1, at least about 0.9:1 and/or not more than about 2:1, not more than about 1.5:1, not more than about 1.1:1, or in the range of from about 0.5:1 to about 2:1, about 0.5:1 to about 1.5:1, about 0.5:1 to about 1.1:1, about 0.75:1 to about 2:1, about 0.75:1 to about 1.5:1, about 0.75:1 to about 1.1:1, about 0.9:1 to about 2:1, about 0.9:1 to about 1.5:1, or about 0.9:1 to about 1.1:1.

The first crystallization cooling period can be performed at a first average crystallization cooling rate, R_{C1}, while the second crystallization cooling period can be performed at a second average crystallization cooling rate, R_{C2}. The first and second average crystallization cooling rates, R_{C1} and R_{C2}, may be different from one another and, embodiments exist wherein R_{C2} can be less (slower) than R_{C1}. Embodiments also exist in which R_{C2} can be greater (faster) than R_{C1}, and embodiments exist in which R_{C2} is approximately equal to R_{C1}. As described above with respect to the average cooling rates of the hydroquinone-containing solution, the average crystallization cooling rate between two crystallization temperatures, T_{CA} and T_{CB}, taken at a first time, t_{A}, and a second time, t_{B}, respectively, can be calculated according to the following formula: (T_{CB}−T_{CA})/(t_{B}−t_{A}). Thus, regardless of the shape of the actual temperature-time curve between T_{CA} and T_{CB}, the average crystallization cooling rate is equal to the slope of a straight line drawn between the two points, T_{CB} and T_{CA}, on a temperature versus time graph.

Embodiments exist in which R_{C1} can be at least about 0.0010, at least about 0.0015, at least about 0.0020, at least 0.0025° C./s and/or not more than about 0.0075, not more than about 0.0050, not more than about 0.0035° C./s, or it can be in the range of from about 0.0010 to about 0.0075° C./s, about 0.0010 to about 0.0050° C./s, about 0.0010 to about 0.0035° C./s, about 0.0015 to about 0.0075° C./s, about 0.0015 to about 0.0050° C./s, about 0.0015 to about 0.0035° C./s, about 0.0020 to about 0.0075° C./s, about 0.0020 to about 0.0050° C./s, about 0.0020 to about 0.0035° C./s, about 0.0025 to about 0.0075° C./s, about 0.0025 to about 0.0050° C./s, or about 0.0025 to about 0.0035° C./s.

Embodiments exist in which R_{C2} can be at least about 0.0010, at least about 0.0015, at least about 0.0020, at least 0.0025° C./s and/or not more than about 0.0075, not more than about 0.0050, not more than about 0.0035° C./s, or it can be in the range of from about 0.0010 to about 0.0075° C./s, about 0.0010 to about 0.0050° C./s, about 0.0010 to about 0.0035° C./s, about 0.0015 to about 0.0075° C./s, about 0.0015 to about 0.0050° C./s, about 0.0015 to about 0.0035° C./s, about 0.0020 to about 0.0075° C./s, about 0.0020 to about 0.0050° C./s, about 0.0020 to about 0.0035° C./s, about 0.0025 to about 0.0075° C./s, about 0.0025 to about 0.0050° C./s, or about 0.0025 to about 0.0035° C./s.

The ratio of the first crystallization cooling rate to the second crystallization cooling rate (R_{C1}: R_{C2}) can be at least about 0.10:1, at least about 0.5:1, at least about 0.75:1, at least about 0.9:1 and/or not more than 3:1, not more than about 2:1, not more than about 1.5:1, not more than about 1.1:1, or in the range of from about 0.1:1 to about 3:1, about 0.1:1 to about 2:1, about 0.1:1 to about 1.5:1, about 0.1:1 to about 1.1:1, about 0.5:1 to about 3:1, about 0.5:1 to about 2:1, about 0.5:1 to about 1.5:1, about 0.5:1 to about 1.1:1, about 0.75:1 to about 3:1, about 0.75:1 to about 2:1, about 0.75:1 to about 1.5:1, about 0.75:1 to about 1.1:1, about 0.9:1 to about 3:1, about 0.9:1 to about 2:1, about 0.9:1 to about 1.5:1, or about 0.9:1 to about 1.1:1. Embodiments exist in which R_{C1}. R_{C2} is less than 1:1, greater than 1:1, or equal to 1:1.

Embodiments exist in which the crystallization slurry in zone 12 is subjected to more than two crystallization cooling periods, one or more of which may have a cooling rate different than, or the same as, one or more others. The cooling of the crystallized hydroquinone slurry may include one or more later crystallization cooling periods having higher (faster) rates of cooling than one or more earlier crystallization cooling periods, or one or more later crystallization cooling periods may have lower (slower) rates of cooling than one or more earlier crystallization cooling periods. In addition, or the alternative, two or more crystallization cooling periods may have approximately the same rate of cooling.

Optionally, the step of further cooling the crystallization slurry from T_{C1} to T_{CF} may include one or more hold periods, during which the temperature and/or pressure of the crystallization slurry may remain substantially constant. For example, the crystallization slurry is cooled from T_{C1} to T_{CF}, it may be subjected to one or more crystallization hold periods, during which the temperature of the slurry is maintained at a relatively constant temperature for a period of time. Any suitable number of crystallization hold periods can be performed as the slurry is cooled from T_{C1} to T_{CF}, and the crystallization hold periods can be carried out at any suitable point in time. Embodiments exist in which the crystallization slurry is subjected to a crystallization hold period between two cooling periods, such that the temperature of the slurry can be maintained within about 5° C., within about 3° C., within about 2° C., within about 1° C., or within about 0.5° C. of one of T_{C1}, T_{C2}, T_{C3}, or any other specified temperature between T_{C1} and T_{CF}. The average cooling rate during a crystallization hold period can be less than 0.0001, less than 0.00005, or less than 0.00001° C./s, measured between the initial hold period temperature and the temperature of the solution at the end of the hold period.

When present, a crystallization hold period can be maintained for a length of time of at least about 15 minutes, at least about 30 minutes, at least about 45 minutes and/or not more than about 2 hours, not more than about 1.5 hours, or not more than about 1 hour, or it can be in the range of from about 15 minutes to about 2 hours, about 15 minutes to about 1.5 hours, about 15 minutes to about 1 hour, about 30 minutes to about 2 hours, about 30 minutes to about 1.5 hours, about 30 minutes to about 1 hour, about 45 minutes to about 2 hours, about 45 minutes to about 1.5 hours, or about 45 minutes to about 1 hour. Embodiments exist in which the ratio of the length of time of at least one crystallization hold period to the length of time of the initial hold period following achievement of the crystallization temperature, T_{C}, is at least about 0.25:1, at least about 0.50:1, at least about 0.75:1, at least about 0.90:1 and/or not more than about 2.5:1, not more than about 2:1, not more than about 1.75:1, not more than about 1.5:1, or not more than about 1.1:1, or the ratio can be in the range of from about 0.25:1 to about 2.5:1, about 0.25:1 to about 2:1, about 0.25:1 to about 1.75:1, about 0.25:1 to about 1.5:1, about 0.25:1 to about 1.1:1, about 0.50:1 to about 2.5:1, about 0.50:1 to about 2:1, about 0.50:1 to about 1.75:1, about 0.50:1 to about 1.5:1, about 0.50:1 to about 1.1:1, about 0.75:1 to about 2.5:1, about 0.75:1 to about 2:1, about 0.75:1 to about 1.75:1, about 0.75:1 to about 1.5:1, about 0.75:1 to about 1.1:1, about 0.90:1 to about 2.5:1, about 0.90:1 to about 2:1, about 0.90:1 to about 1.75:1, about 0.90:1 to about 1.5:1, about 0.90:1 to about 1.1:1. When two or more crystallization hold periods are carried out during the cooling of the crystallization slurry, the lengths of the hold periods may be the same, or one or more may be different.

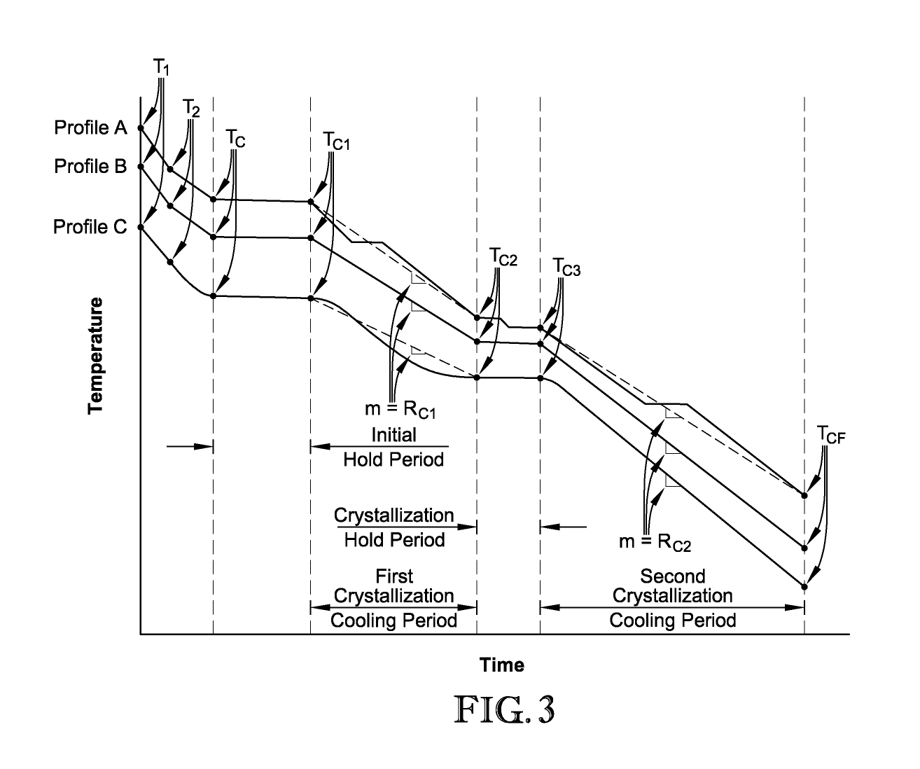

Any suitable number of crystallization cooling stages and/or intermediate crystallization hold periods may be utilized when cooling the crystallization slurry from the first crystallization temperature, T_{C1}, to a final crystallization temperature, T_{CF}. Turning now to FIG. 3, several exemplary cooling profiles, designated as Profiles A through C in FIG. 3, suitable for reducing the temperature of a hydroquinone-containing solution from its initial temperature, T₁, to a final crystallization temperature, T_{CF}, according to various embodiments of the present invention are illustrated.

Although shown in FIG. 3 as being staggered relative to one another in a vertical direction, it should be understood that nothing in FIG. 3 is intended to imply that one or more of Profiles A through C must be conducted at a higher or a lower temperature than any other of Profiles A through C. Rather, the values for temperature, the lengths of various hold periods and cooling periods, and the rates of cooling for the cooling periods for each of Profiles A through C shown in FIG. 3 may vary, and may have values that fall within one or more of the ranges described above. Profiles A through C in FIG. 3 are staggered from one another only to more clearly illustrate the shapes of each curves according to various embodiments of the present invention. Additionally, it should be understood that characteristics of one or more of the profiles shown in FIG. 3 could be combined or cooling profiles could be utilized that include characteristics not specifically illustrated by Profiles A through C without departing from the spirit of the present invention.

As shown in FIG. 3, each of exemplary Profiles A through C may cool the hydroquinone-containing solution from an initial temperature, T₁, to the crystallization temperature, T_{C}, using two initial cooling stages. As shown in the embodiments depicted in FIG. 3, the first cooling stage is carried out to reduce the temperature of the solution from a first initial temperature, T₁, to a second initial temperature, T₂, and the second initial cooling stage shown in FIG. 3 cools the solution from the second initial temperature, T₂, to the crystallization temperature, T_{C}. In the embodiments shown in FIG. 3, the temperature of the solution is then maintained at a substantially constant temperature for an initial hold period. Upon completion of the initial hold period, the crystallization slurry may then be cooled, during a first crystallization cooling period from a first crystallization temperature, T_{C1}, to a second crystallization temperature, T_{C2}, and then from a third crystallization temperature, T_{C3}, to a final crystallization temperature, T_{CF}, after completion of a crystallization hold period.

As shown by comparison of Profiles A through C depicted in FIG. 3, the shape of the actual temperature-time profile during the first and/or second crystallization cooling periods may vary, and may not necessarily be linear, although, as illustrated by Profiles B and C, one or more may be. Embodiments exist in which the temperature of the crystallization solution may be held constant (i.e., a zero or near-zero cooling rate) for at least a portion of the first initial cooling period, as shown by Profile A, and embodiments also exist in which the cooling rate may change instantaneously or nearly instantaneously in order to provide a more curved temperature-time profile, as shown by Profile C. As shown in FIG. 3, the average crystallization cooling rate for each of the crystallization cooling periods, R_{C1} and R_{C2}, of Profiles A through C is equal to the slope, m, of a straight line drawn between the initial and final temperatures of each cooling period (e.g., T_{C1} and T_{C2} for the first crystallization cooling period shown in FIG. 3 and T_{C3} and T_{CF} for the second crystallization cooling period shown in FIG. 3) regardless of the actual shape of the temperature-time profile during the cooling period.

As also shown in FIG. 3, the temperature of the crystallization slurry may change slightly during one or more crystallization hold periods, as long as the temperature remains within about 5° C., within about 3° C., within about 2° C., within about 1° C., or within about 0.5° C. of the specified temperature. For example, as shown in the embodiment depicted by Profile A, the temperature of the crystallization slurry may decrease slightly, or otherwise vary, during the crystallization hold period, as long as it remains within one or more boundaries defined above.

Although shown in FIG. 3 as including two crystallization cooling periods, it should be understood that any suitable number of crystallization cooling periods can be used and, embodiments exist in which a sufficiently large number of crystallization cooling periods may be used such that the cooling profile appears to be a smooth curve. For example, embodiments exist in which the temperature of the hydroquinone-containing slurry being cooled can be within about 5° C., within about 3° C., within about 2° C., or within about 1° C. of a specified or predicted temperature and may follow a time-temperature curve within the above-described limits. Alternatively, or in addition, the actual temperature of the hydroquinone-containing slurry may be within about 40 percent, within about 30 percent, within about 25 percent, or within about 15 percent of the predicted temperature. The predicted temperature, Tₚ, may change, during the course of the further cooling, such that the predicted temperature is dependent on the amount of time, t, that has passed since the initiation of the second, post-crystallization cooling step. In one embodiment, the predicted temperature of the hydroquinone-containing slurry, Tₚ, can be predicted by Equation (3), below:

Tₚ=T_{C}+λt³ (3),

wherein T_{C} is the crystallization temperature and t represents the time since the initiation of the post-crystallization cooling. The coefficient λ in Equation (4) above is the crystallization coefficient, which may be defined by Equation (4), below:

$$λ = ( \frac{{T}_{CF} - {T}_{C}}{Δ {t}^{3}} ) . \qquad ( 4 )$$

In Equations (1) and (2), above, T_{CF} is the final crystallization temperature, T_{C}, is the crystallization temperature, and Δt is the period of time over which the further cooling of the crystallized hydroquinone slurry is performed, beginning with t=0 at the initiation of the cooling of the crystallization slurry. Values for each of these parameters may fall within the ranges provided previously.

The crystallization parameter, λ, can be at least about −9×10⁻⁷, at least about −7.5×10⁻⁷, at least about −5×10⁻⁷° C./s³ and/or not more than about −5×10⁻⁵, not more than about −7.5×10⁻⁵, not more than about −9×10⁻⁵° C./s³, or in the range of from about −9×10⁻⁷ to about −5×10⁻⁵° C./s³, about −9×10⁻⁷ to about −7.5×10⁻⁵° C./s³, about −9×10⁻⁷ to about −9×10⁻⁵° C./s³, about −7.5×10⁻⁷ to about −5×10⁻⁵° C./s³, about −7.5×10⁻⁷ to about −7.5×10⁻⁵° C./s³, about −7.5×10⁻⁷ to about −9×10⁻⁵° C./s³, about −5×10⁻⁷ to about −5×10⁻⁵° C./s³, about −5×10⁻⁷ to about −7.5×10⁻⁵° C./s³, or about −5×10⁻⁷ to about −9×10⁻⁵° C./s³.

Regardless of the specific cooling profile employed, at least some of the hydroquinone originally present in the solution introduced into crystallization zone 12 may have been crystallized to form hydroquinone particles as the crystallization slurry is cooled from T_{C} to T_{CF}. Embodiments exist in which the amount of hydroquinone crystallized out of solution during the cooling from T_{C} to T_{CF} is at least about 75, at least about 80, at least about 85, at least about 90, at least about 95, or at least about 97 percent.

Referring again to FIG. 1, after the crystallization slurry has reached the final crystallization temperature in crystallization zone 12, the slurry may be transported, via conduit 112, to a separation zone 14. Separation zone 14 may be configured to remove at least a portion of the crystallization fluid to thereby provide a separated crystallization fluid and a plurality of wet hydroquinone crystals. Embodiments exist in which separation zone 14 may be operable to remove at least about 20, at least about 30, at least about 40, at least about 50, at least about 60, at least about 70, at least about 80, or at least about 90 percent of the total weight of the crystallization liquid originally present in the slurry introduced into the zone, so that the moisture (or liquid) content of the wet crystals is less than about 50, less than about 45, less than about 40, less than about 35, less than about 30, less than about 25, less than about 20, less than about 10, less than about 8, less than about 6, less than about 5, less than about 2, or less than about 1 weight percent, based on the total weight of the separated crystals.

Optionally, the separated crystals can be contacted with a wash liquid in separation zone 14 to remove impurities, and the washed particles may again be separated from the wash liquid. The wash liquid may have a composition similar to, or different than, the crystallization liquid. For example, embodiments exist in which both the crystallization liquid and wash liquid can be aqueous liquids and may include less than about 5, less than about 2, less than about 1, or less than about 0.5 weight percent of one or more organic components. When utilized, the separation and washing steps, alternated as described above, can be repeated one or more times, depending on the purity of the crystallized hydroquinone slurry and the final product specifications.

Separation zone 14 can comprise one or more solid-liquid separators for separating the hydroquinone crystals from the slurry. Examples of suitable solid-liquid separators can include, but are not limited to, belt filters, rotary filters, centrifugal filters, hydrocyclones, and drum filters. The separation vessels can be operated at or above atmospheric pressure, or can be operated under a vacuum at pressures below atmospheric. When separation zone 14 includes more than one solid-liquid separation vessel, the vessels may be configured in parallel or in series and can be operated in a batch, semi-batch, or continuous mode.

Referring again to FIG. 1, the separated crystals withdrawn from separation zone 14 via conduit 114 may then be routed to a drying zone 16, wherein the particles may be dried to provide a plurality of dried particles. The drying can be carried out for a time period of at least about 5, at least about 10, at least about 15 minutes and/or not more than about 1 hour, not more than about 45, or not more than about 30 minutes, or a time period in the range of from about 5 minutes to about 1 hour, about 5 to about 45 minutes, about 5 to about 30 minutes, about 10 minutes to about 1 hour, about 10 to about 45 minutes, about 10 to about 30 minutes, about 15 minutes to about 1 hour, about 15 to about 45 minutes, or about 15 to about 30 minutes.

In one embodiment, the drying carried out in drying zone 16 can be performed by reducing the pressure of all or a portion of drying zone 16. Alternatively, or in addition, the particles may be heated within drying zone 16 via direct or indirect heat exchange with a heat transfer fluid. As used herein in reference to heat exchange, the term “direct” means that the material being heated or cooled comes into direct contact with the heat transfer medium, while “indirect” means that the material being heated or cooled and/or the heat transfer medium may contact an intermediate element, such as, for example, the walls of a heat exchanger tube or the walls of the dryer, which then transfers heat from one to the other. During the drying step, the particles can achieve a maximum temperature, measured in the dryer atmosphere surrounding the particle surfaces, of at least about 65, at least about 70, at least about 75, at least about 78° C. and/or not more than about 105, not more than about 100, not more than about 95, not more than about 90, or not more than about 87° C., or in the range of from about 65 to about 105° C., about 65 to about 100° C., about 65 to about 95° C., about 65 to about 90° C., about 65 to about 87° C., about 70 to about 105° C., about 70 to about 100° C., about 70 to about 95° C., about 70 to about 90° C., about 70 to about 87° C., about 75 to about 105° C., about 75 to about 100° C., about 75 to about 95° C., about 75 to about 90° C., about 75 to about 87° C., about 78 to about 105° C., about 78 to about 100° C., about 78 to about 95° C., about 78 to about 90° C., or about 78 to about 87° C. Drying can be carried out in any suitable type of dryer, which may be operated in a batch, semi-batch, or continuous mode. The moisture content of the dried particles exiting drying zone 16 via conduit 116 can be less than about 2, less than about 1, or less than about 0.5 weight percent.

Referring again to FIG. 1, the particles exiting drying zone 16, shown by conduit 116 in FIG. 1, may be then optionally be transported to a cooling zone 18. In one embodiment, at least a portion of cooling zone 18 can be disposed within drying zone 16 such as, for example, when all or a portion of said cooling is performed within the dryer. In one embodiment, the cooling and drying zones may be separate, such that the drying and cooling of the hydroquinone particles can be performed in separate vessels and the dried particles in conduit 116 can be transported from drying zone 16 to cooling zone 18. When drying zone 16 and cooling zone 18 are separate, at least a portion of the cooling can be performed during the transporting of the dried particles from the drying zone, such that at least a portion of the cooling and transporting are performed simultaneously.

The cooling can be sufficient to reduce the temperature of the particles, measured with at least one temperature indicator, such that at least some, or at least about 50, at least about 60, at least about 75, at least about 80 volume percent of the total amount of particles within cooling zone can be cooled to a temperature of less than about 70, less than about 65, less than about 60, less than about 55, less than about 50, less than about 45, less than about 40, less than about 35, less than about 30, less than about 25, or less than about 20° C. The temperature indicator used to measure the temperature of the cooled particles can be an indirect temperature indicator configured to measure the ambient temperature of the environment surrounding the particle surfaces, or it can be a direct temperature indicator configured to measure the temperature of a collection of several particle surfaces.

The cooling of the particles may be performed by any suitable method. In one embodiment, at least a portion of the cooling may be performed by direct heat exchange, such that a cooling medium, such as air, may come into direct contact with at least a portion of the dried particles. In one embodiment, at least a portion of the cooling may be carried out in a heat exchange device via indirect heat exchange with one or more heat transfer fluids, such as, for example, chilled water.

The particles exiting drying zone 16 and/or cooling zone 18 may have an elongated, approximate needle-like shape, with a median length-to-width, or aspect, ratio (L:W) of greater than 1.5:1 or greater than about 2:1. The median L:W of a representative sample of the cooled particles, as measured by optical microscopy, can be at least about 2.5:1, at least about 3:1, at least about 5:1 and/or not more than about 100:1, not more than about 50:1, not more than about 30:1, not more than about 25:1, not more than about 20:1, or not more than about 15:1, or in the range of from about 2:1 to about 100:1, about 2:1 to about 50:1, about 2:1 to about 30:1, about 2:1 to about 25:1, about 2:1 to about 20:1, about 2:1 to about 15:1, about 2.5:1 to about 100:1, about 2.5:1 to about 50:1, about 2.5:1 to about 30:1, about 2.5:1 to about 25:1, about 2.5:1 to about 20:1, about 2.5:1 to about 15:1, about 3:1 to about 100:1, about 3:1 to about 50:1, about 3:1 to about 30:1, about 3:1 to about 25:1, about 3:1 to about 20:1, about 3:1 to about 15:1, about 5:1 to about 100:1, about 5:1 to about 50:1, about 5:1 to about 30:1, about 5:1 to about 25:1, about 5:1 to about 20:1, or about 5:1 to about 15:1. The aspect ratio of the particles can be measured as described in Example 5, below, or an equivalent method.

The number-averaged aspect ratio distribution of a representative sample of the crystallized hydroquinone particles can have a median aspect ratio, or A50, of at least about 2.95:1, at least about 3:1, at least about 3.25:1, at least about 3.5:1, at least about 3.75:1, at least about 4:1, at least about 4.25:1, at least about 4.5:1, at least about 4.75:1, at least about 5:1, at least about 5.25:1, at least about 5.3:1, at least about 5.35:1 and/or not more than about 6.5:1, not more than about 6.25:1, not more than about 6:1, or not more than about 5.75:1.

The median aspect ratio, A50, of a representative sample of the hydroquinone particles exiting drying zone 16 and/or cooling zone 18 can be in the range of from about 2.95:1 to about 6.5:1, about 2.95:1 to about 6.25:1, about 2.95:1 to about 6:1, about 2.95:1 to about 5.75:1, about 3:1 to about 6.5:1, about 3:1 to about 6.25:1, about 3:1 to about 6:1, about 3:1 to about 5.75:1, about 3.25:1 to about 6.5:1, about 3.25:1 to about 6.25:1, about 3.25:1 to about 6:1, about 3.25:1 to about 5.75:1, about 3.5:1 to about 6.5:1, about 3.5:1 to about 6.25:1, about 3.5:1 to about 6:1, about 3.5:1 to about 5.75:1, about 3.75:1 to about 6.5:1, about 3.75:1 to about 6.25:1, about 3.75:1 to about 6:1, about 3.75:1 to about 5.75:1, about 4:1 to about 6.5:1, about 4:1 to about 6.25:1, about 4:1 to about 6:1, about 4:1 to about 5.75:1, about 4.25:1 to about 6.5:1, about 4.25:1 to about 6.25:1, about 4.25:1 to about 6:1, about 4.25:1 to about 5.75:1, about 4.5:1 to about 6.5:1, about 4.5:1 to about 6.25:1, about 4.5:1 to about 6:1, about 4.5:1 to about 5.75:1, about 4.75:1 to about 6.5:1, about 4.75:1 to about 6.25:1, about 4.75:1 to about 6:1, about 4.75:1 to about 5.75:1, about 5:1 to about 6.5:1, about 5:1 to about 6.25:1, about 5:1 to about 6:1, about 5:1 to about 5.75:1, about 5.25:1 to about 6.5:1, about 5.25:1 to about 6.25:1, about 5.25:1 to about 6:1, about 5.25:1 to about 5.75:1, about 5.3:1 to about 6.5:1, about 5.3:1 to about 6.25:1, about 5.3:1 to about 6:1, about 5.3:1 to about 5.75:1, about 5.35:1 to about 6.5:1, about 5.35:1 to about 6.25:1, about 5.35:1 to about 6:1, or about 5.35:1 to about 5.75:1.

The number-averaged aspect ratio distribution of a representative sample of particles exiting drying zone 16 and/or cooling zone 18 can have a tenth percentile aspect ratio, or A10, of at least about 1.75:1, at least about 1.90:1, at least about 2:1, at least about 2.25:1, at least about 2.25:1, at least about 2.5:1, at least about 2.6:1, at least about 2.7:1, at least about 2.75:1, at least about 2.8:1, at least about 2.85:1 and/or not more than about 3.25:1, not more than about 3.1:1, not more than about 3:1, or not more than about 2.95:1. The number-averaged aspect ratio distribution of a representative sample of particles can have a ninetieth percentile aspect ratio, or A90, of at least about 5.25:1, at least about 5.5:1, at least about 6:1, at least about 6.5:1, at least about 6.5:1, at least about 7:1, at least about 7.5:1, at least about 8:1, at least about 8.5:1, at least about 9:1, at least about 9.5:1, at least about 9.55:1, at least about 9.60:1, at least about 9.65:1 and/or not more than about 11:1, not more than about 10.5:1, not more than about 10:1, or not more than about 9.75:1.

As used herein, the “tenth percentile aspect ratio” or “A10” refers to the aspect ratio under which ten percent of the total distribution lies, based on the total number of measurements. In other words, ten percent of the total number of measurements making up the distribution has an aspect ratio of not more than the A10 value, while ninety percent, based on the total number of measurements, of the sample has an aspect ratio greater than the A10 value. Similarly, the “ninetieth percentile aspect ratio” or “A90” refers to the aspect ratio under which ninety percent of the total number of measurements making up the distribution lies, such that ninety percent of the total number of samples measured has an aspect ratio of not more than the A90, while 10 percent of the sample has an aspect ratio greater than the A90.

The tenth percentile aspect ratio, or A10, for a representative sample of the crystallized hydroquinone particles can be in the range of from about 1.75:1 to about 3.25:1, about 1.75:1 to about 3.1:1, about 1.75:1 to about 3:1, about 1.75:1 to about 2.95:1, about 1.9:1 to about 3.25:1, about 1.9:1 to about 3.1:1, about 1.9:1 to about 3:1, about 1.9:1 to about 2.95:1, about 2:1 to about 3.25:1, about 2:1 to about 3.1:1, about 2:1 to about 3:1, about 2:1 to about 2.95:1, about 2.1:1 to about 3.25:1, about 2.1:1 to about 3.1:1, about 2.1:1 to about 3:1, about 2.1:1 to about 2.95:1, about 2.25:1 to about 3.25:1, about 2.25:1 to about 3.1:1, about 2.25:1 to about 3:1, about 2.25:1 to about 2.95:1, about 2.5:1 to about 3.25:1, about 2.5:1 to about 3.1:1, about 2.5:1 to about 3:1, about 2.5:1 to about 2.95:1, about 2.6:1 to about 3.25:1, about 2.6:1 to about 3.1:1, about 2.6:1 to about 3:1, about 2.6:1 to about 2.95:1, about 2.7:1 to about 3.25:1, about 2.7:1 to about 3.1:1, about 2.7:1 to about 3:1, about 2.7:1 to about 2.95:1, about 2.75:1 to about 3.25:1, about 2.75:1 to about 3.1:1, about 2.75:1 to about 3:1, about 2.75:1 to about 2.95:1, about 2.8:1 to about 3.25:1, about 2.8:1 to about 3.1:1, about 2.8:1 to about 3:1, about 2.8:1 to about 2.95:1, about 2.85:1 to about 3.25:1, about 2.85:1 to about 3.1:1, about 2.85:1 to about 3:1, or about 2.85:1 to about 2.95:1.

The ninetieth percentile aspect ratio, A90, for the aspect ratio distribution of a representative sample of the crystallized hydroquinone particle can be in the range of from about 5.25:1 to about 11:1, about 5.25:1 to about 10.5:1, about 5.25:1 to about 10:1, about 5.25:1 to about 9.75:1, about 5.5:1 to about 11:1, about 5.5:1 to about 10.5:1, about 5.5:1 to about 10:1, about 5.5:1 to about 9.75:1, about 6:1 to about 11:1, about 6:1 to about 10.5:1, about 6:1 to about 10:1, about 6:1 to about 9.75:1, about 6.5:1 to about 11:1, about 6.5:1 to about 10.5:1, about 6.5:1 to about 10:1, about 6.5:1 to about 9.75:1, about 7:1 to about 11:1, about 7:1 to about 10.5:1, about 7:1 to about 10:1, about 7:1 to about 9.75:1, about 7.5:1 to about 11:1, about 7.5:1 to about 10.5:1, about 7.5:1 to about 10:1, about 7.5:1 to about 9.75:1, about 8:1 to about 11:1, about 8:1 to about 10.5:1, about 8:1 to about 10:1, about 8:1 to about 9.75:1, about 8.5:1 to about 11:1, about 8.5:1 to about 10.5:1, about 8.5:1 to about 10:1, about 8.5:1 to about 9.75:1, about 9:1 to about 11:1, about 9:1 to about 10.5:1, about 9:1 to about 10:1, about 9:1 to about 9.75:1, about 9.5:1 to about 11:1, about 9.5:1 to about 10.5:1, about 9.5:1 to about 10:1, about 9.5:1 to about 9.75:1, about 9.55:1 to about 11:1, about 9.55:1 to about 10.5:1, about 9.55:1 to about 10:1, about 9.55:1 to about 9.75:1, about 9.6:1 to about 11:1, about 9.6:1 to about 10.5:1, about 9.6:1 to about 10:1, about 9.6:1 to about 9.75:1, about 9.65:1 to about 11:1, about 9.65:1 to about 10.5:1, about 9.65:1 to about 10:1, or about 9.65:1 to about 9.75:1.

The span of the aspect ratio distribution of a representative sample of the crystallized hydroquinone particles can be at least about 1.18, at least about 1.20, at least about 1.22, at least about 1.25 and/or not more than about 1.32, not more than about 1.30, not more than about 1.27, not more than about 1.25, or in the range of from about 1.18 to about 1.32, about 1.18 to about 1.30, about 1.18 to about 1.27, about 1.18 to about 1.25, about 1.20 to about 1.32, about 1.20 to about 1.30, about 1.20 to about 1.27, about 1.20 to about 1.25, about 1.22 to about 1.32, about 1.22 to about 1.30, about 1.22 to about 1.27, about 1.22 to about 1.25, about 1.25 to about 1.32, about 1.25 to about 1.30, or about 1.25 to about 1.27.

A representative sample of particles exiting drying zone 16 and/or cooling zone 18 may also have a number-averaged length distribution having a median length, or L50, in one or more ranges below. As used herein, the term “length” refers to the longest dimension of a particle. The median length, L50, of a representative sample of the crystallized hydroquinone particles can be at least about 150, at least about 175, at least about 250, at least about 300 microns and/or not more than about 5000, not more than about 3500, not more than about 2500, not more than about 2250, not more than about 2000, not more than about 1750, not more than about 1500 not more than about 1400, not more than about 1300, or not more than about 1250 microns, or it can be in the range of from about 150 to about 5000 microns, about 150 to about 3500 microns, about 150 to about 2500 microns, about 150 to about 2250 microns, about 150 to about 2000 microns, about 150 to about 1750 microns, about 150 to about 1500 microns, about 150 to about 1400 microns, about 150 to about 1300 microns, about 150 to about 1250 microns, about 175 to about 5000 microns, about 175 to about 3500 microns, about 175 to about 2500 microns, about 175 to about 2250 microns, about 175 to about 2000 microns, about 175 to about 1750 microns, about 175 to about 1500 microns, about 175 to about 1400 microns, about 175 to about 1300 microns, about 175 to about 1250 microns, about 250 to about 5000 microns, about 250 to about 3500 microns, about 250 to about 2500 microns, about 250 to about 2250 microns, about 250 to about 2000 microns, about 250 to about 1750 microns, about 250 to about 1500 microns, about 250 to about 1400 microns, about 250 to about 1300 microns, about 250 to about 1250 microns, about 300 to about 5000 microns, about 300 to about 3500 microns, about 300 to about 2500 microns, about 300 to about 2250 microns, about 300 to about 2000 microns, about 300 to about 1750 microns, about 300 to about 1500 microns, about 300 to about 1400 microns, about 300 to about 1300 microns, or about 300 to about 1250 microns.

Embodiments exist in which the median length, L50, of a representative sample of the crystallized hydroquinone particles can be at least about 235, at least about 250, at least about 300, at least about 350, at least about 375, at least about 400, at least about 410, at least about 425, at least about 435, at least about 450, at least about 460, at least about 475, at least about 485, at least about 500, at least about 505 microns and/or not more than about 600, not more than about 550, not more than about 525, or not more than about 515 microns.

The median length, L50, can be in the range of from about 235 to about 600 microns, about 235 to about 550 microns, about 235 to about 525 microns, about 235 to about 515 microns, about 250 to about 600 microns, about 250 to about 550 microns, about 250 to about 525 microns, about 250 to about 515 microns, about 300 to about 600 microns, about 300 to about 550 microns, about 300 to about 525 microns, about 300 to about 515 microns, about 350 to about 600 microns, about 350 to about 550 microns, about 350 to about 525 microns, about 350 to about 515 microns, about 375 to about 600 microns, about 375 to about 550 microns, about 375 to about 525 microns, about 375 to about 515 microns, about 385 to about 600 microns, about 385 to about 550 microns, about 385 to about 525 microns, about 385 to about 515 microns, about 400 to about 600 microns, about 400 to about 550 microns, about 400 to about 525 microns, about 400 to about 515 microns, about 410 to about 600 microns, about 410 to about 550 microns, about 410 to about 525 microns, about 410 to about 515 microns, about 425 to about 600 microns, about 425 to about 550 microns, about 425 to about 525 microns, about 425 to about 515 microns, about 435 to about 600 microns, about 435 to about 550 microns, about 435 to about 525 microns, about 435 to about 515 microns, about 450 to about 600 microns, about 450 to about 550 microns, about 450 to about 525 microns, about 450 to about 515 microns, about 460 to about 600 microns, about 460 to about 550 microns, about 460 to about 525 microns, about 460 to about 515 microns, about 475 to about 600 microns, about 475 to about 550 microns, about 475 to about 525 microns, about 475 to about 515 microns, about 480 to about 600 microns, about 480 to about 550 microns, about 480 to about 525 microns, about 480 to about 515 microns, about 500 to about 600 microns, about 500 to about 550 microns, about 500 to about 525 microns, about 500 to about 515 microns, about 505 to about 600 microns, about 505 to about 550 microns, about 505 to about 525 microns, or about 505 to about 515 microns. The number-averaged length distribution of a representative sample of particles exiting drying zone 16 and/or cooling zone 18 can have a tenth percentile length, or L10, of at least about 100, at least about 125, at least about 135, at least about 140, at least about 145, at least about 150, at least about 155, at least about 160, at least about 165 microns and/or not more than about 200, not more than about 190, not more than about 175 microns. The number-averaged length distribution of a representative sample of particles exiting drying zone 16 and/or cooling zone 18 can have a ninetieth percentile length, or L90, of at least about 550, at least about 600, at least about 650, at least about 700, at least about 750, at least about 790, at least about 800, at least about 810, at least about 820, at least about 830, at least about 840, at least about 850, at least about 860, at least about 870, at least about 880, at least about 890, at least about 900, at least about 910, at least about 920, at least about 930, at least about 940, at least about 950, at least about 960, at least about 970, at least about 980, at least about 990, at least about 1000, at least about 1010, at least about 1020, at least about 1030, at least about 1040, at least about 1050, at least about 1060, at least about 1065 microns and/or not more than about 1200, not more than about 1150, not more than about 1100, not more than about 1080 microns.

As used herein, the “tenth percentile length” or “L10” refers to the length under which ten percent of the total distribution lies, based on the total number of measurements. In other words, ten percent of the total number of measurements making up the distribution has a length of not more than the L10 value, while ninety percent, based on the total number of measurements, of the sample has a length greater than the L10 value. Similarly, the “ninetieth percentile length” or “L90” refers to the length under which ninety percent of the total number of measurements making up the distribution lies, such that ninety percent of the total measured particles has an aspect ratio of not more than the L90, while 10 percent of the measured particles has an aspect ratio greater than the L90 value.

The tenth percentile length, or L10, for a representative sample of the crystallized hydroquinone particles can be in the range of from about 100 to about 200 microns, about 100 to about 190 microns, about 100 to about 175 microns, about 125 to about 200 microns, about 125 to about 190 microns, about 125 to about 175 microns, about 135 to about 200 microns, about 135 to about 190 microns, about 135 to about 175 microns, about 140 to about 200 microns, about 140 to about 190 microns, about 140 to about 175 microns, about 145 to about 200 microns, about 145 to about 190 microns, about 145 to about 175 microns, about 150 to about 200 microns, about 150 to about 190 microns, about 150 to about 175 microns, about 155 to about 200 microns, about 155 to about 190 microns, about 155 to about 175 microns, about 160 to about 200 microns, about 160 to about 190 microns, about 160 to about 175 microns, about 165 to about 200 microns, about 165 to about 190 microns, or about 165 to about 175 microns.

The ninetieth percentile length, or L90, for a representative sample of the crystallized hydroquinone particles can be in the range of from about 550 to about 1200 microns, about 550 to about 1150 microns, about 550 to about 1100 microns, about 550 to about 1080 microns, about 600 to about 1200 microns, about 600 to about 1150 microns, about 600 to about 1100 microns, about 600 to about 1080 microns, about 650 to about 1200 microns, about 650 to about 1150 microns, about 650 to about 1100 microns, about 650 to about 1080 microns, about 700 to about 1200 microns, about 700 to about 1150 microns, about 700 to about 1100 microns, about 700 to about 1080 microns, about 750 to about 1200 microns, about 750 to about 1150 microns, about 750 to about 1100 microns, about 750 to about 1080 microns, about 790 to about 1200 microns, about 790 to about 1150 microns, about 790 to about 1100 microns, about 790 to about 1080 microns, about 800 to about 1200 microns, about 800 to about 1150 microns, about 800 to about 1100 microns, about 800 to about 1080 microns, about 810 to about 1200 microns, about 810 to about 1150 microns, about 810 to about 1100 microns, about 810 to about 1080 microns, about 820 to about 1200 microns, about 820 to about 1150 microns, about 820 to about 1100 microns, about 820 to about 1080 microns, about 830 to about 1200 microns, about 830 to about 1150 microns, about 830 to about 1100 microns, about 830 to about 1080 microns, about 840 to about 1200 microns, about 840 to about 1150 microns, about 840 to about 1100 microns, about 840 to about 1080 microns, about 850 to about 1200 microns, about 850 to about 1150 microns, about 850 to about 1100 microns, about 850 to about 1080 microns, about 860 to about 1200 microns, about 860 to about 1150 microns, about 860 to about 1100 microns, about 860 to about 1080 microns, about 870 to about 1200 microns, about 870 to about 1150 microns, about 870 to about 1100 microns, about 870 to about 1080 microns, about 880 to about 1200 microns, about 880 to about 1150 microns, about 880 to about 1100 microns, about 880 to about 1080 microns, about 890 to about 1200 microns, about 890 to about 1150 microns, about 890 to about 1100 microns, about 890 to about 1080 microns, about 900 to about 1200 microns, about 900 to about 1150 microns, about 900 to about 1100 microns, about 900 to about 1080 microns, about 910 to about 1200 microns, about 910 to about 1150 microns, about 910 to about 1100 microns, about 910 to about 1080 microns, about 920 to about 1200 microns, about 920 to about 1150 microns, about 920 to about 1100 microns, about 920 to about 1080 microns, about 930 to about 1200 microns, about 930 to about 1150 microns, about 930 to about 1100 microns, about 930 to about 1080 microns, about 940 to about 1200 microns, about 940 to about 1150 microns, about 940 to about 1100 microns, about 940 to about 1080 microns about 950 to about 1200 microns, about 950 to about 1150 microns, about 950 to about 1100 microns, about 950 to about 1080 microns, about 960 to about 1200 microns, about 960 to about 1150 microns, about 960 to about 1100 microns, about 960 to about 1080 microns, about 970 to about 1200 microns, about 970 to about 1150 microns, about 970 to about 1100 microns, about 970 to about 1080 microns, about 980 to about 1200 microns, about 980 to about 1150 microns, about 980 to about 1100 microns, about 980 to about 1080 microns, about 990 to about 1200 microns, about 990 to about 1150 microns, about 990 to about 1100 microns, about 990 to about 1080 microns, about 1000 to about 1200 microns, about 1000 to about 1150 microns, about 1000 to about 1100 microns, about 1000 to about 1080 microns, about 1010 to about 1200 microns, about 1010 to about 1150 microns, about 1010 to about 1100 microns, about 1010 to about 1080 microns, about 1020 to about 1200 microns, about 1020 to about 1150 microns, about 1020 to about 1100 microns, about 1020 to about 1080 microns, about 1030 to about 1200 microns, about 1030 to about 1150 microns, about 1030 to about 1100 microns, about 1030 to about 1080 microns, about 1040 to about 1200 microns, about 1040 to about 1150 microns, about 1040 to about 1100 microns, about 1040 to about 1080 microns, about 1050 to about 1200 microns, about 1050 to about 1150 microns, about 1050 to about 1100 microns, about 1050 to about 1080 microns, about 1060 to about 1200 microns, about 1060 to about 1150 microns, about 1060 to about 1100 microns, about 1060 to about 1080 microns, about 1065 to about 1200 microns, about 1065 to about 1150 microns, about 1065 to about 1100 microns, or about 1065 to about 1080 microns.

The volume-averaged particle size distribution of a representative sample of particles exiting drying zone 16 and/or cooling zone 18 can have a median particle size, or Dv50, of at least about 250, at least about 260, at least about 265, at least about 270, at least about 275, at least about 280, at least about 285, at least about 290, at least about 295, at least about 300, at least about 305, at least about 310, at least about 315 and/or not more than about 340, not more than about 335, not more than about 330, not more than about 325, not more than about 320, not more than about 315, or not more than about 310 microns, as measured by a laser scattering technique performed on a 2-gram sample of the particles in a dry cell using a Horiba LA950 Laser Scattering Particle Size Distribution Analyzer (available from Horiba Scientific in Kyoto, Japan), with an air pressure of 0.3 MPa, a laser wavelength of 655 nm, and a refractive index of 1.689-0.010i, or an equivalent method.

The representative sample may be withdrawn from a larger composition of hydroquinone particles. Embodiments exist in which the representative sample of particles may be withdrawn from a composition comprising at least about 5, at least about 10, at least about 15, at least about 25, at least about 50, at least about 100, at least about 250, at least about 400, at least about 500, at least about 750, at least about 1000 kg of hydroquinone particles, which may optionally be contained within a space defined by a storage container, as discussed in further detail below. Embodiments exist in which the crystallized hydroquinone particles can comprise at least about 50, at least about 60, at least about 70, at least about 80, at least about 90, at least about 95, at least about 98, at least about 99, or 100 percent by weight of the total composition. The composition may consist essentially of, or consist of, crystallized hydroquinone particles.

The median particle size (Dv50), which is measured as the diameter of a sphere having equivalent volume (equivalent spherical diameter), of the representative sample can be in the range of from about 250 to about 340 microns, about 250 to about 335 microns, about 250 to about 330 microns, about 250 to about 325 microns, about 250 to about 320 microns, about 250 to about 315 microns, about 250 to about 310 microns, about 260 to about 340 microns, about 260 to about 335 microns, about 260 to about 330 microns, about 260 to about 325 microns, about 260 to about 320 microns, about 260 to about 315 microns, about 260 to about 310 microns, about 265 to about 340 microns, about 265 to about 335 microns, about 265 to about 330 microns, about 265 to about 325 microns, about 265 to about 320 microns, about 265 to about 315 microns, about 265 to about 310 microns, about 270 to about 340 microns, about 270 to about 335 microns, about 270 to about 330 microns, about 270 to about 325 microns, about 270 to about 320 microns, about 270 to about 315 microns, about 270 to about 310 microns, about 275 to about 340 microns, about 275 to about 335 microns, about 275 to about 330 microns, about 275 to about 325 microns, about 275 to about 320 microns, about 275 to about 315 microns, about 275 to about 310 microns, about 280 to about 340 microns, about 280 to about 335 microns, about 280 to about 330 microns, about 280 to about 325 microns, about 280 to about 320 microns, about 280 to about 315 microns, about 280 to about 310 microns, about 285 to about 340 microns, about 285 to about 335 microns, about 285 to about 330 microns, about 285 to about 325 microns, about 285 to about 320 microns, about 285 to about 315 microns, about 285 to about 310 microns, about 290 to about 340 microns, about 290 to about 335 microns, about 290 to about 330 microns, about 290 to about 325 microns, about 290 to about 320 microns, about 290 to about 315 microns, about 290 to about 310 microns, about 295 to about 340 microns, about 295 to about 335 microns, about 295 to about 330 microns, about 295 to about 325 microns, about 295 to about 320 microns, about 295 to about 315 microns, about 295 to about 310 microns, about 300 to about 340 microns, about 300 to about 335 microns, about 300 to about 330 microns, about 300 to about 325 microns, about 300 to about 320 microns, about 300 to about 315 microns, about 300 to about 310 microns, about 305 to about 340 microns, about 305 to about 335 microns, about 305 to about 330 microns, about 305 to about 325 microns, about 305 to about 320 microns, about 305 to about 315 microns, about 305 to about 310 microns, about 310 to about 340 microns, about 310 to about 335 microns, about 310 to about 330 microns, about 310 to about 325 microns, about 310 to about 320 microns, about 310 to about 315 microns, about 315 to about 340 microns, about 315 to about 335 microns, about 315 to about 330 microns, about 315 to about 325 microns, or about 315 to about 320 microns.

The particle size distribution a representative sample of the crystallized hydroquinone particles may also have a tenth percentile particle size, or Dv10, measured as the equivalent spherical diameter, of at least about 115, at least about 120, at least about 125, at least about 130, at least about 135, at least about 140 microns and/or not more than about 150, not more than about 145, not more than about 140, not more than about 135, not more than about 132, not more than about 130, not more than about 127, or not more than about 125 microns.

The particle size distribution of a representative sample of the crystallized hydroquinone particles may also have a ninetieth percentile particle size, or Dv90, measured as the equivalent spherical diameter, of at least about 675, at least about 680, at least about 690, at least about 700, at least about 710, at least about 720, at least about 730, at least about 740, at least about 750, at least about 760, at least about 770, at least about 780, at least about 790, at least about 800, at least about 810, at least about 820, at least about 830, at least about 835 microns and/or not more than about 890, not more than about 875, not more than about 860, not more than about 850, not more than about 840, not more than about 830, not more than about 825, not more than about 820, not more than about 815, not more than about 810, or not more than about 805 microns, also measured by laser diffraction.

As used herein, the “tenth percentile particle size” or “Dv10” refers to the particle size under which ten volume percent of the total distribution lies. In other words, ten percent of the total volume of the sample has a particle size of not more than the Dv10 value, while ninety volume percent of the sample has a particle size greater than the Dv10 value. Similarly, the “ninetieth percentile particle size” or “Dv90” refers to the particle size under which ninety volume percent of the total volume of the distribution lies, such that ninety volume percent of the total sample has a particle size of not more than the Dv90, while 10 volume percent of the sample has a particle size greater than the Dv90.

The Dv10 of the representative particle size distribution can be in the range of from about 115 to about 150 microns, about 115 to about 145 microns, about 115 to about 140 microns, about 115 to about 135 microns, about 115 to about 132 microns, about 115 to about 130 microns, about 115 to about 127 microns, about 115 to about 125 microns, about 120 to about 150 microns, about 120 to about 145 microns, about 120 to about 140 microns, about 120 to about 135 microns, about 120 to about 132 microns, about 120 to about 130 microns, about 120 to about 127 microns, about 120 to about 125 microns, about 125 to about 150 microns, about 125 to about 145 microns, about 125 to about 140 microns, about 125 to about 135 microns, about 125 to about 132 microns, about 125 to about 130 microns, about 125 to about 127 microns, about 125 to about 125 microns, about 130 to about 150 microns, about 130 to about 145 microns, about 130 to about 140 microns, about 130 to about 135 microns, about 130 to about 132 microns, about 130 to about 130 microns, about 130 to about 127 microns, about 130 to about 125 microns, about 135 to about 150 microns, about 135 to about 145 microns, about 135 to about 140 microns, about 140 to about 150 microns, or about 140 to about 145 microns.

The Dv90 of the representative particle size distribution can be in the range of from about 675 to about 890 microns, about 675 to about 875 microns, about 675 to about 860 microns, about 675 to about 850 microns, about 675 to about 840 microns, about 675 to about 830 microns, about 675 to about 825 microns, about 675 to about 820 microns, about 675 to about 815 microns, about 675 to about 810 microns, about 675 to about 805 microns, about 680 to about 890 microns, about 680 to about 875 microns, about 680 to about 860 microns, about 680 to about 850 microns, about 680 to about 840 microns, about 680 to about 830 microns, about 680 to about 825 microns, about 680 to about 820 microns, about 680 to about 815 microns, about 680 to about 810 microns, about 680 to about 805 microns, about 690 to about 890 microns, about 690 to about 875 microns, about 690 to about 860 microns, about 690 to about 850 microns, about 690 to about 840 microns, about 690 to about 830 microns, about 690 to about 825 microns, about 690 to about 820 microns, about 690 to about 815 microns, about 690 to about 810 microns, about 690 to about 805 microns, about 700 to about 890 microns, about 700 to about 875 microns, about 700 to about 860 microns, about 700 to about 850 microns, about 700 to about 840 microns, about 700 to about 830 microns, about 700 to about 825 microns, about 700 to about 820 microns, about 700 to about 815 microns, about 700 to about 810 microns, about 700 to about 805 microns, about 710 to about 890 microns, about 710 to about 875 microns, about 710 to about 860 microns, about 710 to about 850 microns, about 710 to about 840 microns, about 710 to about 830 microns, about 710 to about 825 microns, about 710 to about 820 microns, about 710 to about 815 microns, about 710 to about 810 microns, about 710 to about 805 microns, about 720 to about 890 microns, about 720 to about 875 microns, about 720 to about 860 microns, about 720 to about 850 microns, about 720 to about 840 microns, about 720 to about 830 microns, about 720 to about 825 microns, about 720 to about 820 microns, about 720 to about 815 microns, about 720 to about 810 microns, about 720 to about 805 microns, about 730 to about 890 microns, about 730 to about 875 microns, about 730 to about 860 microns, about 730 to about 850 microns, about 730 to about 840 microns, about 730 to about 830 microns, about 730 to about 825 microns, about 730 to about 820 microns, about 730 to about 815 microns, about 730 to about 810 microns, about 730 to about 805 microns, about 740 to about 890 microns, about 740 to about 875 microns, about 740 to about 860 microns, about 740 to about 850 microns, about 740 to about 840 microns, about 740 to about 830 microns, about 740 to about 825 microns, about 740 to about 820 microns, about 740 to about 815 microns, about 740 to about 810 microns, about 740 to about 805 microns, about 750 to about 890 microns, about 750 to about 875 microns, about 750 to about 860 microns, about 750 to about 850 microns, about 750 to about 840 microns, about 750 to about 830 microns, about 750 to about 825 microns, about 750 to about 820 microns, about 750 to about 815 microns, about 750 to about 810 microns, about 750 to about 805 microns, about 760 to about 890 microns, about 760 to about 875 microns, about 760 to about 860 microns, about 760 to about 850 microns, about 760 to about 840 microns, about 760 to about 830 microns, about 760 to about 825 microns, about 760 to about 820 microns, about 760 to about 815 microns, about 760 to about 810 microns, about 760 to about 805 microns, about 770 to about 890 microns, about 770 to about 875 microns, about 770 to about 860 microns, about 770 to about 850 microns, about 770 to about 840 microns, about 770 to about 830 microns, about 770 to about 825 microns, about 770 to about 820 microns, about 770 to about 815 microns, about 770 to about 810 microns, about 770 to about 805 microns, about 780 to about 890 microns, about 780 to about 875 microns, about 780 to about 860 microns, about 780 to about 850 microns, about 780 to about 840 microns, about 780 to about 830 microns, about 780 to about 825 microns, about 780 to about 820 microns, about 780 to about 815 microns, about 780 to about 810 microns, about 780 to about 805 microns, about 790 to about 890 microns, about 790 to about 875 microns, about 790 to about 860 microns, about 790 to about 850 microns, about 790 to about 840 microns, about 790 to about 830 microns, about 790 to about 825 microns, about 790 to about 820 microns, about 790 to about 815 microns, about 790 to about 810 microns, about 790 to about 805 microns, about 800 to about 890 microns, about 800 to about 875 microns, about 800 to about 860 microns, about 800 to about 850 microns, about 800 to about 840 microns, about 800 to about 830 microns, about 800 to about 825 microns, about 800 to about 820 microns, about 800 to about 815 microns, about 800 to about 810 microns, about 800 to about 805 microns, about 805 to about 890 microns, about 805 to about 875 microns, about 805 to about 860 microns, about 805 to about 850 microns, about 805 to about 840 microns, about 805 to about 830 microns, about 805 to about 825 microns, about 805 to about 820 microns, about 805 to about 815 microns, about 805 to about 810 microns, about 810 to about 890 microns, about 810 to about 875 microns, about 810 to about 860 microns, about 810 to about 850 microns, about 810 to about 840 microns, about 810 to about 830 microns, about 810 to about 825 microns, about 810 to about 820 microns, about 810 to about 815 microns, about 820 to about 890 microns, about 820 to about 875 microns, about 820 to about 860 microns, about 820 to about 850 microns, about 820 to about 840 microns, about 820 to about 830 microns, about 820 to about 825 microns, about 830 to about 890 microns, about 830 to about 875 microns, about 830 to about 860 microns, about 830 to about 850 microns, about 830 to about 840 microns, about 840 to about 890 microns, about 840 to about 875 microns, about 840 to about 860 microns, or about 840 to about 850 microns.

The representative particle size distribution can have a span of at least about 1.5, at least about 1.6, at least about 1.7, at least about 1.75, at least about 1.8, at least about 1.9, at least about 2.0, at least about 2.05, at least about 2.1, at least about 2.15, at least about 2.2, at least about 2.25, at least about 2.3 and/or not more than about 2.75, not more than about 2.65, not more than about 2.5, not more than about 2.4, not more than about 2.35, not more than about 2.3, not more than about 2.25, not more than about 2.2, not more than about 2.15, not more than about 2.1, or not more than about 2.05. As used herein, the term “span,” used with respect to particle size distribution, is calculated according to the following equation: (Dv90−Dv10)/(Dv50).

The span of the particle size distribution of the representative sample of the crystallized hydroquinone particles can be in the range of from about 1.5 to about 2.75, about 1.5 to about 2.65, about 1.5 to about 2.5, about 1.5 to about 2.4, about 1.5 to about 2.35, about 1.5 to about 2.3, about 1.5 to about 2.25, about 1.5 to about 2.2, about 1.5 to about 2.15, about 1.5 to about 2.1, about 1.5 to about 2.05, about 1.6 to about 2.75, about 1.6 to about 2.65, about 1.6 to about 2.5, about 1.6 to about 2.4, about 1.6 to about 2.35, about 1.6 to about 2.3, about 1.6 to about 2.25, about 1.6 to about 2.2, about 1.6 to about 2.15, about 1.6 to about 2.1, about 1.6 to about 2.05, about 1.7 to about 2.75, about 1.7 to about 2.65, about 1.7 to about 2.5, about 1.7 to about 2.4, about 1.7 to about 2.35, about 1.7 to about 2.3, about 1.7 to about 2.25, about 1.7 to about 2.2, about 1.7 to about 2.15, about 1.7 to about 2.1, about 1.7 to about 2.05, about 1.75 to about 2.75, about 1.75 to about 2.65, about 1.75 to about 2.5, about 1.75 to about 2.4, about 1.75 to about 2.35, about 1.75 to about 2.3, about 1.75 to about 2.25, about 1.75 to about 2.2, about 1.75 to about 2.15, about 1.75 to about 2.1, about 1.75 to about 2.05, about 1.8 to about 2.75, about 1.8 to about 2.65, about 1.8 to about 2.5, about 1.8 to about 2.4, about 1.8 to about 2.35, about 1.8 to about 2.3, about 1.8 to about 2.25, about 1.8 to about 2.2, about 1.8 to about 2.15, about 1.8 to about 2.1, about 1.8 to about 2.05, about 1.9 to about 2.75, about 1.9 to about 2.65, about 1.9 to about 2.5, about 1.9 to about 2.4, about 1.9 to about 2.35, about 1.9 to about 2.3, about 1.9 to about 2.25, about 1.9 to about 2.2, about 1.9 to about 2.15, about 1.9 to about 2.1, about 1.9 to about 2.05, about 2.0 to about 2.75, about 2.0 to about 2.65, about 2.0 to about 2.5, about 2.0 to about 2.4, about 2.0 to about 2.35, about 2.0 to about 2.3, about 2.0 to about 2.25, about 2.0 to about 2.2, about 2.0 to about 2.15, about 2.0 to about 2.1, or about 2.0 to about 2.05, about 2.05 to about 2.75, about 2.05 to about 2.65, about 2.05 to about 2.5, about 2.05 to about 2.4, about 2.05 to about 2.35, about 2.05 to about 2.3, about 2.05 to about 2.25, about 2.05 to about 2.2, about 2.05 to about 2.15, about 2.05 to about 2.1, about 2.1 to about 2.75, about 2.1 to about 2.65, about 2.1 to about 2.5, about 2.1 to about 2.4, about 2.1 to about 2.35, about 2.1 to about 2.3, about 2.1 to about 2.25, about 2.1 to about 2.2, about 2.1 to about 2.15, about 2.15 to about 2.75, about 2.15 to about 2.65, about 2.15 to about 2.5, about 2.15 to about 2.4, about 2.15 to about 2.35, about 2.15 to about 2.3, about 2.15 to about 2.25, about 2.15 to about 2.2, about 2.2 to about 2.75, about 2.2 to about 2.65, about 2.2 to about 2.5, about 2.2 to about 2.4, about 2.2 to about 2.35, about 2.2 to about 2.3, about 2.2 to about 2.25, about 2.25 to about 2.75, about 2.25 to about 2.65, about 2.25 to about 2.5, about 2.25 to about 2.4, about 2.25 to about 2.35, about 2.25 to about 2.3, about 2.3 to about 2.75, about 2.3 to about 2.65, about 2.3 to about 2.5, about 2.3 to about 2.4, or about 2.3 to about 2.35.

The average crystallite size of a representative sample of hydroquinone particles exiting drying zone 16 and/or cooling zone 18 can be at least about 375, at least about 400, at least about 450, at least about 500, at least about 525, at least about 550, at least about 600, at least about 650, at least about 675 angstroms and/or not more than about 850, not more than about 800, not more than about 750, not more than about 700, not more than about 650, or not more than about 600 angstroms, measured at a 2-theta angle of 9.5° for the 220 reflection of the powder XRD analysis performed using a copper anode X-ray tube. The average crystallite size of the representative sample of particles may be in the range of from about 375 to about 850 angstroms, about 375 to about 800 angstroms, about 375 to about 750 angstroms, about 375 to about 700 angstroms, about 375 to about 650 angstroms, about 375 to about 600 angstroms, about 400 to about 850 angstroms, about 400 to about 800 angstroms, about 400 to about 750 angstroms, about 400 to about 700 angstroms, about 400 to about 650 angstroms, about 400 to about 600 angstroms, about 450 to about 850 angstroms, about 450 to about 800 angstroms, about 450 to about 750 angstroms, about 450 to about 700 angstroms, about 450 to about 650 angstroms, about 450 to about 600 angstroms, about 500 to about 850 angstroms, about 500 to about 800 angstroms, about 500 to about 750 angstroms, about 500 to about 700 angstroms, about 500 to about 650 angstroms, about 500 to about 600 angstroms, about 525 to about 850 angstroms, about 525 to about 800 angstroms, about 525 to about 750 angstroms, about 525 to about 700 angstroms, about 525 to about 650 angstroms, about 525 to about 600 angstroms, about 550 to about 850 angstroms, about 550 to about 800 angstroms, about 550 to about 750 angstroms, about 550 to about 700 angstroms, about 550 to about 650 angstroms, about 550 to about 600 angstroms, about 600 to about 850 angstroms, about 600 to about 800 angstroms, about 600 to about 750 angstroms, about 600 to about 700 angstroms, about 600 to about 650 angstroms, about 650 to about 850 angstroms, about 650 to about 800 angstroms, about 650 to about 750 angstroms, about 650 to about 700 angstroms, about 675 to about 850 angstroms, about 675 to about 800 angstroms, about 675 to about 750 angstroms, or about 675 to about 700 angstroms.

A representative sample of the hydroquinone particles exiting drying zone 16 and/or cooling zone 18 may have a crystalline melting temperature (Tₘ), measured on a DSC first heating scan at a rate of 20° C./s, of at least about 170.5, at least about 171, at least about 171.5, at least about 172, at least about 172.5, at least about 173° C. and/or not more than about 175, not more than about 174.5, not more than about 174, or not more than about 173.75° C., or in the range of from about 170.5 to about 175° C., about 170.5 to about 174.5° C., about 170.5 to about 174° C., about 170.5 to about 173.75° C., about 171 to about 175° C., about 171 to about 174.5° C., about 171 to about 174° C., about 171 to about 173.75° C., about 171.5 to about 175° C., about 171.5 to about 174.5° C., about 171.5 to about 174° C., about 171.5 to about 173.75° C., about 172 to about 175° C., about 172 to about 174.5° C., about 172 to about 174° C., about 172 to about 173.75° C., about 172.5 to about 175° C., about 172.5 to about 174.5° C., about 172.5 to about 174° C., about 172.5 to about 173.75° C., about 173 to about 175° C., about 173 to about 174.5° C., about 173 to about 174° C., or about 173 to about 173.75° C.

The heat of fusion of a representative sample of the particles, measured on the first DSC scan, can be at least about 58, at least about 59, at least about 59.5, at least about 60, at least about 60.5, at least about 61 cal/g and/or not more than about 63, not more than about 62.5, not more than about 62, not more than about 61.75, not more than about 61, or not more than about 60.75 cal/g, or it can be in the range of from about 58 to about 63 cal/g, about 58 to about 62.5 cal/g, about 58 to about 62 cal/g, about 58 to about 61.75 cal/g, about 58 to about 61 cal/g, about 58 to about 60.75 cal/g, about 59 to about 63 cal/g, about 59 to about 62.5 cal/g, about 59 to about 62 cal/g, about 59 to about 61.75 cal/g, about 59 to about 61 cal/g, about 59 to about 60.75 cal/g, about 59.5 to about 63 cal/g, about 59.5 to about 62.5 cal/g, about 59.5 to about 62 cal/g, about 59.5 to about 61.75 cal/g, about 59.5 to about 61 cal/g, about 59.5 to about 60.75 cal/g, about 60 to about 63 cal/g, about 60 to about 62.5 cal/g, about 60 to about 62 cal/g, about 60 to about 61.75 cal/g, about 60 to about 61 cal/g, about 60 to about 60.75 cal/g, about 60.5 to about 63 cal/g, about 60.5 to about 62.5 cal/g, about 60.5 to about 62 cal/g, about 60.5 to about 61.75 cal/g, about 60.5 to about 61 cal/g, about 60.5 to about 60.75 cal/g, about 61 to about 63 cal/g, about 61 to about 62.5 cal/g, about 61 to about 62 cal/g, or about 61 to about 61.75 cal/g.

The specific surface area of a representative sample of the particles exiting drying zone 16 and/or cooling zone 18 can be at least about 0.035, at least about 0.040, at least about 0.045, at least about 0.050, or at least about 0.055 m²/g and/or not more than about 0.075, not more than about 0.070, not more than about 0.065, not more than about 0.055, or not more than about 0.050 m²/g, measured using a BET surface adsorption technique performed on a Micromeritics ASAP 2020 instrument and ASAP 2020 analysis software (available from Micromeritics Corporation, Norcross, Ga., USA) with Krypton as the adsorbate at 77K and with 10-second equilibration intervals, after sample has been degassed for 12 hours at 50° C., or an equivalent method.

The specific surface area may be in the range of from about 0.035 to about 0.075 m²/g, about 0.035 to about 0.070 m²/g, about 0.035 to about 0.065 m²/g, about 0.035 to about 0.055 m²/g, about 0.035 to about 0.050 m²/g, about 0.040 to about 0.075 m²/g, about 0.040 to about 0.070 m²/g, about 0.040 to about 0.065 m²/g, about 0.040 to about 0.055 m²/g, about 0.040 to about 0.050 m²/g, about 0.045 to about 0.075 m²/g, about 0.045 to about 0.070 m²/g, about 0.045 to about 0.065 m²/g, about 0.045 to about 0.055 m²/g, about 0.045 to about 0.050 m²/g, about 0.050 to about 0.075 m²/g, about 0.050 to about 0.070 m²/g, about 0.050 to about 0.065 m²/g, about 0.050 to about 0.055 m²/g, about 0.055 to about 0.075 m²/g, about 0.055 to about 0.070 m²/g, about 0.055 to about 0.065 m²/g.

The specific surface area may be in the range of from about 0.045 to about 0.10 m²/g, about 0.045 to about 0.080 m²/g, about 0.045 to about 0.075 m²/g, about 0.045 to about 0.070 m²/g, about 0.045 to about 0.065 m²/g, about 0.050 to about 0.10 m²/g, about 0.050 to about 0.080 m²/g, about 0.050 to about 0.075 m²/g, about 0.050 to about 0.070 m²/g, about 0.050 to about 0.065 m²/g, about 0.055 to about 0.10 m²/g, about 0.055 to about 0.080 m²/g, about 0.055 to about 0.075 m²/g, about 0.055 to about 0.070 m²/g, about 0.055 to about 0.065 m²/g, about 0.060 to about 0.10 m²/g, about 0.060 to about 0.080 m²/g, about 0.060 to about 0.075 m²/g, about 0.060 to about 0.070 m²/g, or about 0.060 to about 0.065 m²/g.

The crystallized hydroquinone particles may have a bulk density of not more than about 0.90, not more than about 0.85, not more than about 0.80, not more than about 0.75, not more than about 0.70, not more than about 0.65, not more than about 0.64, not more than about 0.63, not more than about 0.62, not more than about 0.61, not more than about 0.60, not more than about 0.59, not more than about 0.58, not more than about 0.57, not more than about 0.56, not more than about 0.55, not more than about 0.54, not more than about 0.53, not more than about 0.52, not more than about 0.51, not more than about 0.50 g/cm³, as measured under ambient conditions with an FT4 Powder Rheometer System (available from Freeman Technology, Tewkesbury, UK) located indoors under ambient temperature and humidity using 75 grams of material in the shear cell test (using an applied normal stress of 9 kPa), or with an equivalent method.

The bulk density of the representative sample of the crystallized hydroquinone particles can be at least about 0.40, at least about 0.42, at least about 0.44, at least about 0.46, at least about 0.48, at least about 0.50 g/cm³ and/or not more than about 0.65, not more than about 0.60, not more than about 0.55, not more than about 0.54, or not more than about 0.52 g/cm³, or in the range of from about 0.40 to about 0.65 g/cm³, about 0.40 to about 0.60 g/cm³, about 0.40 to about 0.55 g/cm³, about 0.40 to about 0.54 g/cm³, about 0.40 to about 0.52 g/cm³, about 0.42 to about 0.65 g/cm³, about 0.42 to about 0.60 g/cm³, about 0.42 to about 0.55 g/cm³, about 0.42 to about 0.54 g/cm³, about 0.42 to about 0.52 g/cm³, about 0.44 to about 0.65 g/cm³, about 0.44 to about 0.60 g/cm³, about 0.44 to about 0.55 g/cm³, about 0.44 to about 0.54 g/cm³, about 0.44 to about 0.52 g/cm³, about 0.46 to about 0.65 g/cm³, about 0.46 to about 0.60 g/cm³, about 0.46 to about 0.55 g/cm³, about 0.46 to about 0.54 g/cm³, about 0.46 to about 0.52 g/cm³, about 0.48 to about 0.65 g/cm³, about 0.48 to about 0.60 g/cm³, about 0.48 to about 0.55 g/cm³, about 0.48 to about 0.54 g/cm³, about 0.48 to about 0.52 g/cm³, about 0.50 to about 0.65 g/cm³, about 0.50 to about 0.60 g/cm³, about 0.50 to about 0.55 g/cm³, about 0.50 to about 0.54 g/cm³, or about 0.50 to about 0.52 g/cm³.

Turning again to FIG. 1, the particles exiting drying zone 16 and/or cooling zone 18 in conduit 118 can then be routed to a loading zone 20, as shown in FIG. 1. In loading zone 20, the cooled particles can be loaded into one or more shipping containers, whereafter the loaded containers can be transported out of facility 10 and on to further storage, shipping, and/or use. Containers can be formed of any suitable material, not incompatible with the particles, and may include, for example, drums, totes, bags, sacks, supersacks, or combinations thereof. Embodiments exist in which the weight of crystallized hydroquinone particles loaded into a container can be at least about 5, at least about 10, at least about 20, at least about 50, at least about 100, at least about 150, at least about 200, at least about 250, at least about 400, at least about 500, at least about 750, at least about 1000, at least about 1500 kg.

After loading, the loaded container may be shipped across a transport path from one location to another. In one embodiment, at least a portion of the transport path may cross at least one ocean. Transportation may be provided by one or more suitable modes, including, but not limited to, truck, rail car, and ship and, the total transport and/or storage time can be at least about 1 day, at least about 5 days, at least about 1 week, at least about 2 weeks and/or not more than 1 year, not more than about 6 months, not more than about 2 months, not more than about 1 month, or not more than about 3 weeks, or a period of time in the range of from about 1 day to about 1 year, about 1 day to about 6 months, about 2 months, about 1 day to about 1 month, 1 day to about 3 weeks, about 5 days to about 1 year, about 5 days to about 6 months, about 5 days to about 2 months, about 5 days to about 1 month, 5 days to about 3 weeks, about 1 week to about 1 year, about 1 week to about 6 months, about 2 months, about 1 week to about 1 month, 1 week to about 3 weeks, about 2 weeks to about 1 year, about 2 weeks to about 6 months, about 2 weeks to about 2 months, about 2 weeks to about 1 month, or about 2 weeks to about 3 weeks. Upon unloading the particles from the container, it may be found that the cooled, shipped particles can have substantially the same values for one or more of the above-discussed properties within the ranges discussed above. As used herein, the term “substantially the same” means within 5 percent of one or more of the values or ranges provided above. For example, an unloaded particle having substantially the same median particle size would have a median particle size within 5 percent of the median particle size of the cooled particles loaded into the shipping container. Thus, upon unloading at a destination, hydroquinone particles produced according to embodiments of the present invention may have substantially the same particle size, particle size distribution, crystallite size, surface area, melting point, and fines content as the cooled particles loaded into the container in loading zone 20 of facility 10 shown in FIG. 1.

### EXAMPLES

### Example 1

### Crystallization Cooling Curves

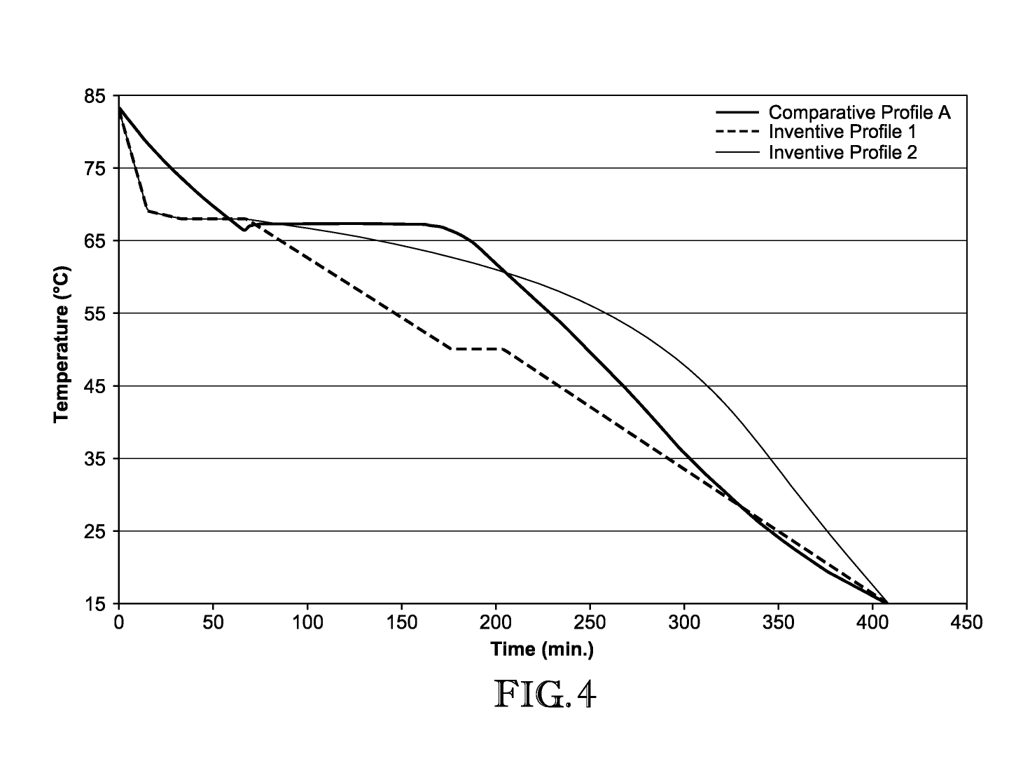

Three crystallization cooling profiles were used to cool hydroquinone-containing solutions using evaporative cooling from a crystallization temperature of approximately 66.5 to 67° C. to a final crystallization temperature of 15° C. Each of the profiles, including Comparative Profile A, Inventive Profile 1, and Inventive Profile 2, is represented graphically in FIG. 4 and Tables 1-3 below provide the temperatures and the approximate cooling rates after a given time for each of the profiles illustrated in FIG. 4. The cooling rates provided in Table 1, below, were calculated according the formula provided above.

TABLE 1
Cooling Rates and Temperatures for
Comparative Cooling Profile A

| Time (t), min | Temperature (T), ° C. | Cooling Rate, ° C./s |
|---|---|---|
| 0 | 67 | — |
| 102.4 | 65 | 0.020 |
| 204.8 | 57.5 | 0.073 |
| 242 | 39.2 | 0.492 |
| 261 | 34.1 | 0.266 |
| 280 | 29.7 | 0.232 |
| 301 | 25.5 | 0.201 |
| 320 | 22.6 | 0.152 |
| 347 | 18.3 | 0.079 |

TABLE 2
Cooling Rates and Temperatures for Inventive Cooling Profile 1

| Time (t), min | Temperature (T), ° C. | Cooling Rate, ° C./s |
|---|---|---|
| 0 | 67 | — |
| 34.1 | 67 | 0 |
| 143.4 | 50 | 0.156 |
| 170.7 | 50 | 0 |
| 374 | 15 | 0.172 |

TABLE 3
Cooling Rates and Temperatures for Inventive Cooling Profile 2

| Time (t), min | Temperature (T), ° C. | Cooling Rate, ° C./s |
|---|---|---|
| 0 | 67 | — |
| 34.1 | 67 | 0 |
| 102.4 | 65 | 0.029 |
| 204.8 | 57.5 | 0.073 |
| 273.1 | 46.5 | 0.161 |
| 374.0 | 15 | 0.312 |

As shown in the Tables above and in FIG. 4, Comparative Profile A included a low initial cooling rate of 0.020° C., which was gradually increased to a maximum rate of about 0.49° C./s, before again being reduced to a final cooling rate of 0.079° C./s. Inventive Cooling Profile 1 included an initial 30-minute hold at 67° C., followed by a period of cooling at a rate of 0.015° C./s, which was then followed by another 30-minute hold at a temperature of 50° C. The final cooling stage of Inventive Profile 1 was performed at nearly the same cooling rate as the first stage (0.172° C./s). Inventive Cooling Profile 2 also included an initial 30-minute hold at 67° C., followed by cooling rates that became progressively higher until the final temperature of about 15° C. was reached. All profiles had an overall cooling time of about 6 hours and achieved a final temperature of about 15° C.

### Example 2

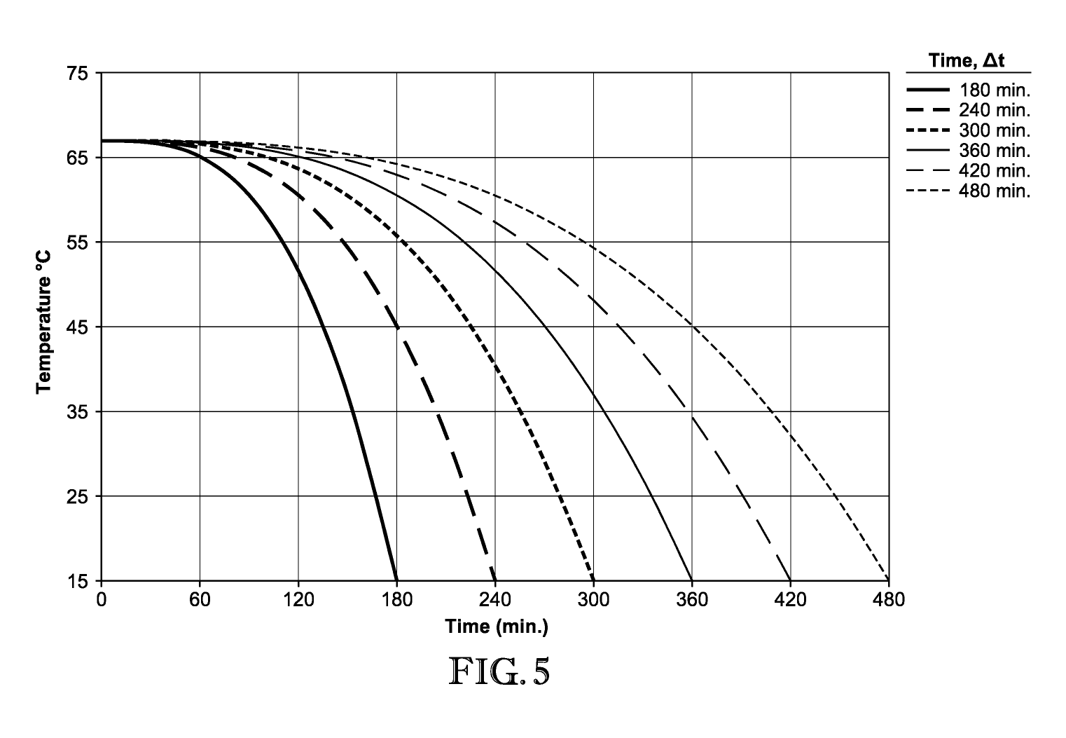

Several exemplary crystallization cooling curves, suitable for cooling a crystallized hydroquinone-solution from a crystallization temperature, T_{C}, of 67° C. to a final crystallization temperature, T_{CF}, of 15° C., were modeled and are shown in FIG. 5. Each of the time-dependent temperature curves shown in FIG. 5 are characterized by the formula provided in Equation (3), above, and vary from one another according to the time period, Δt, over which the cooling was modeled. The time periods for the cooling curves show in FIG. 5 range from 3 hours (180 minutes) to 8 hours (480 minutes). Table 4, below, summarizes the value of the crystallization coefficient, λ, defined according to Equation 2, above, for each of the cooling curves shown in FIG. 5.

TABLE 4
Values for Crystallization Coefficient
(λ) for Cooling Curves shown in FIG. 5

| Time Period, Δt | Crystallization Coefficient, λ |
|---|---|
| (min) | (° C./s³) |
| 180 | −8.92 × 10⁻⁶ |
| 240 | −3.76 × 10⁻⁶ |
| 300 | −1.93 × 10⁻⁶ |
| 360 | −1.11 × 10⁻⁶ |
| 420 | −7.02 × 10⁻⁷ |
| 480 | −4.70 × 10⁻⁷ |

Upon application of the above cooling curves to a hydroquinone-containing crystallization slurry, the actual temperature of the slurry would be expected stay within the temperature predicted by Equations (1) and (2), provided above, according to one or more ranges described previously.

### Example 3

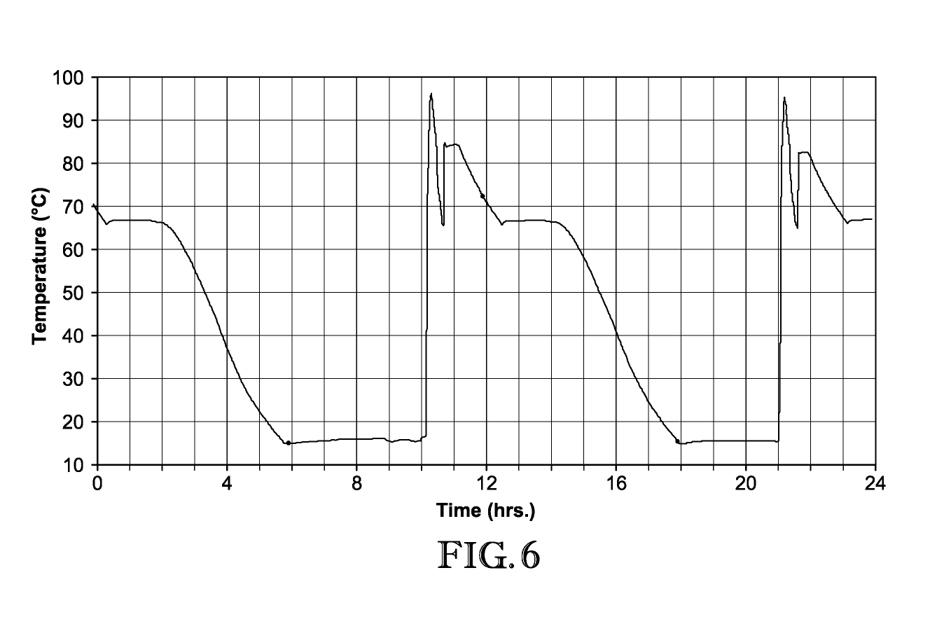

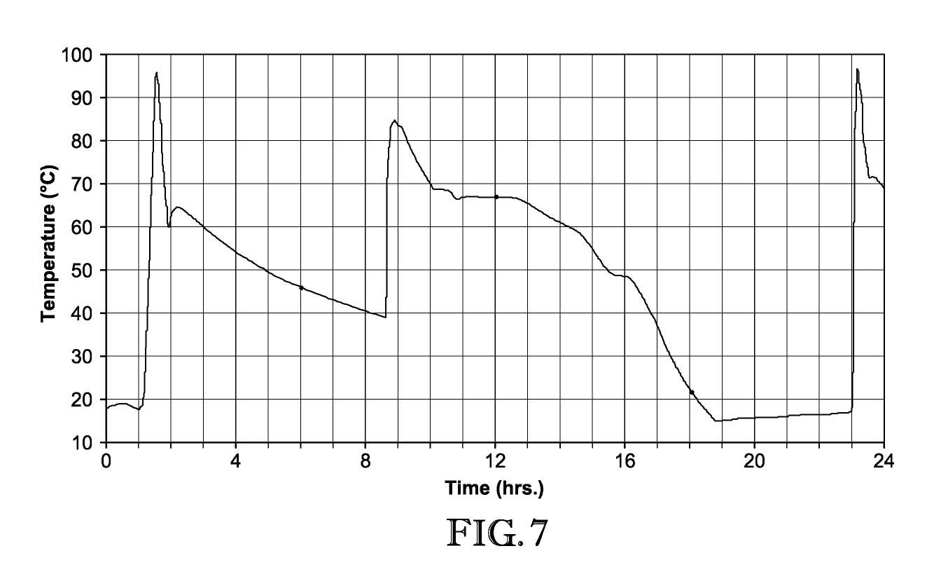

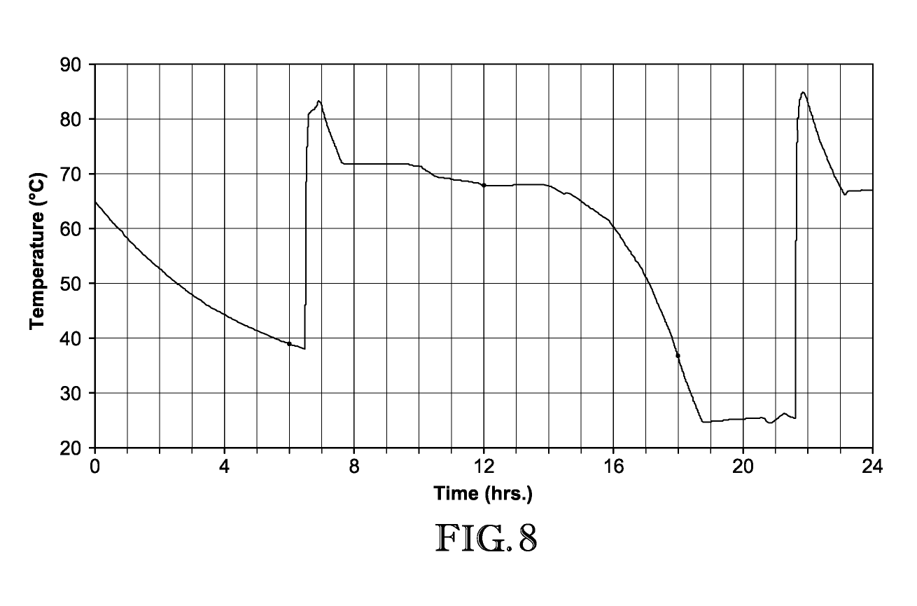

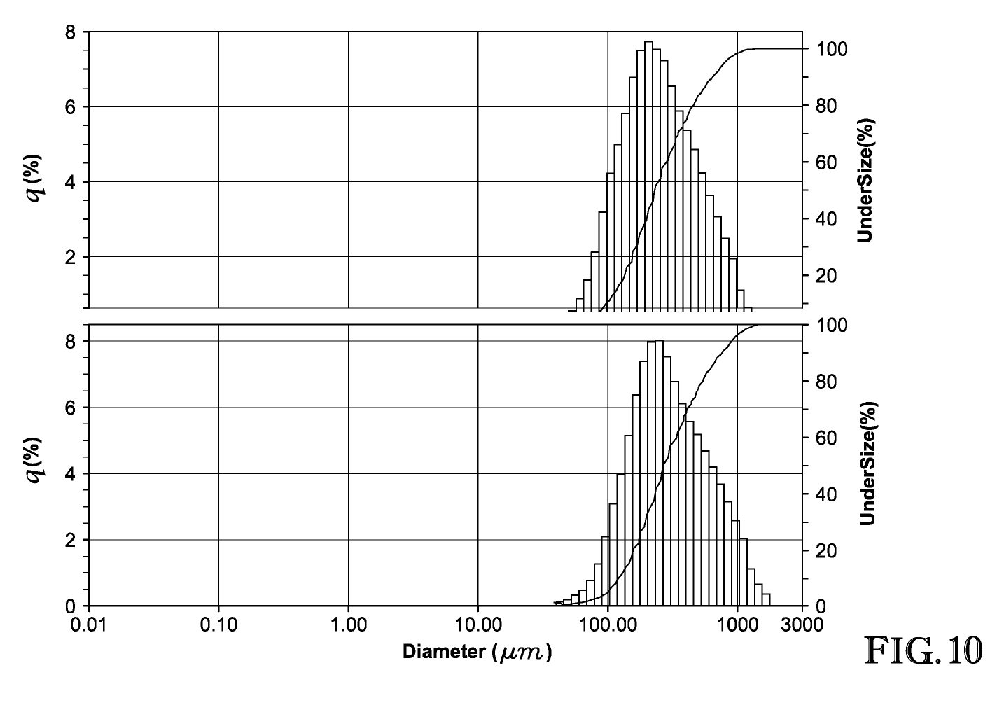

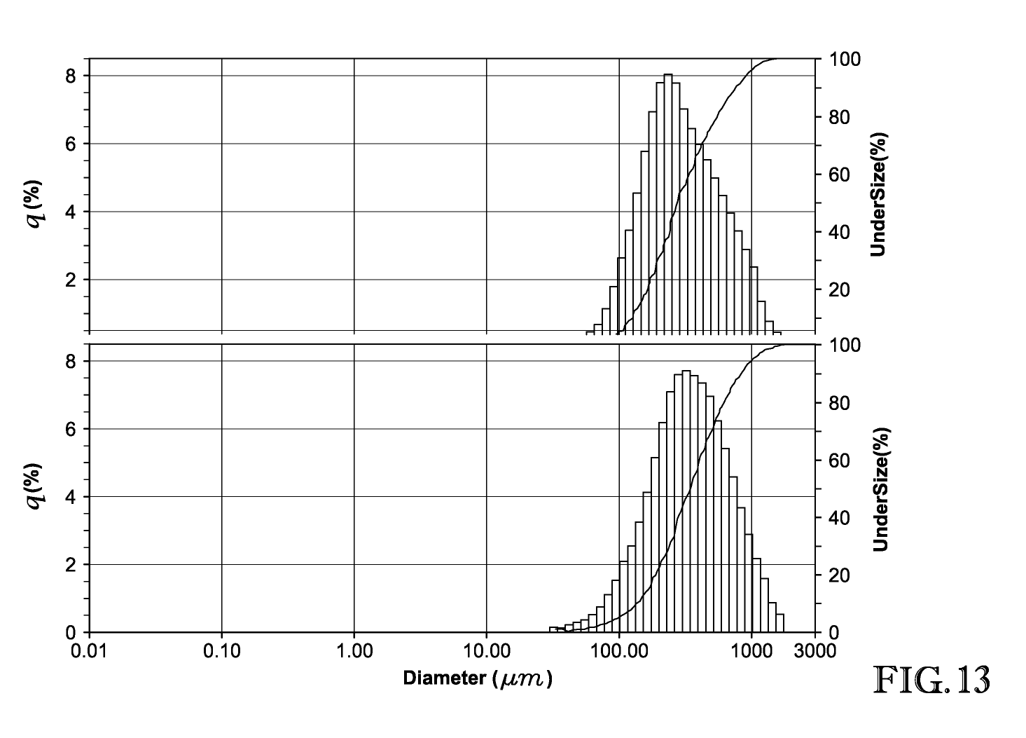

Four separate batches of hydroquinone-containing solution were cooled and crystallized according to the crystallization profiles illustrated in FIGS. 6-9. FIG. 6 was a comparative cooling profile (Comparative Profile B), while FIGS. 7-9 represented inventive profiles (Inventive Profiles 3-5). After crystallization, the particles were dried and recovered and analyzed for particle size distribution. The particle size distributions were determined using a Horiba Laser Scattering Particle Size Distribution Analyzer (commercially available from Horiba Scientific in Kyoto, Japan) according to the above-described method. Graphical depictions of the particle size distributions of the crystals formed using Inventive Profiles 3-5 are shown in the lower portion of the PSD graphs in FIGS. 10-12, while the PSD graph of the crystals formed with Comparative Profile B are provided in the upper portion of each of FIGS. 10-12. Additionally, FIG. 13 compares the particle size distribution of the hydroquinone particles crystallized according Inventive Profile 5, shown in FIG. 8, as compared to another sample of conventionally-prepared hydroquinone. Selected information related to each of the particle size distributions provided in FIGS. 10-13 is provided in Table 5, below.

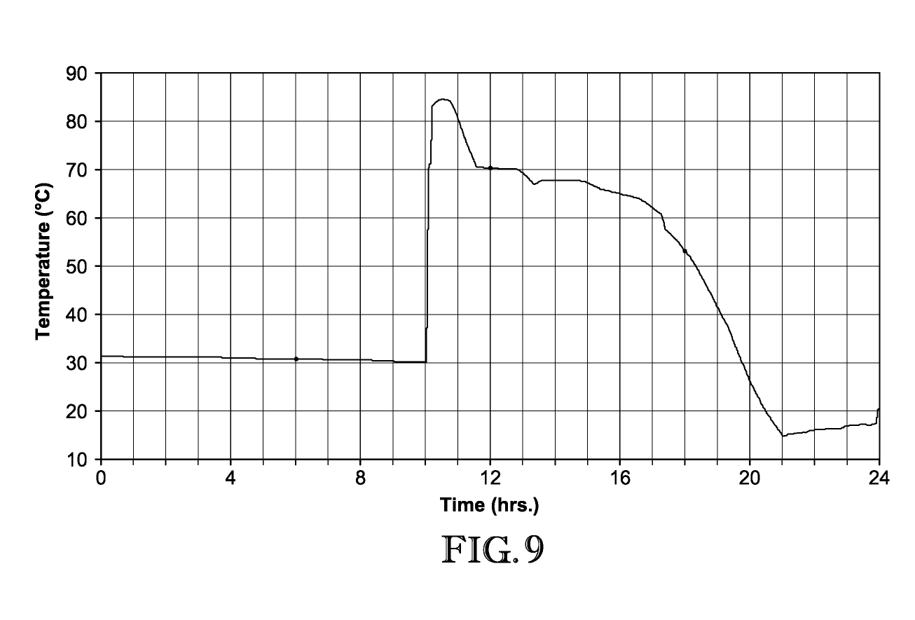

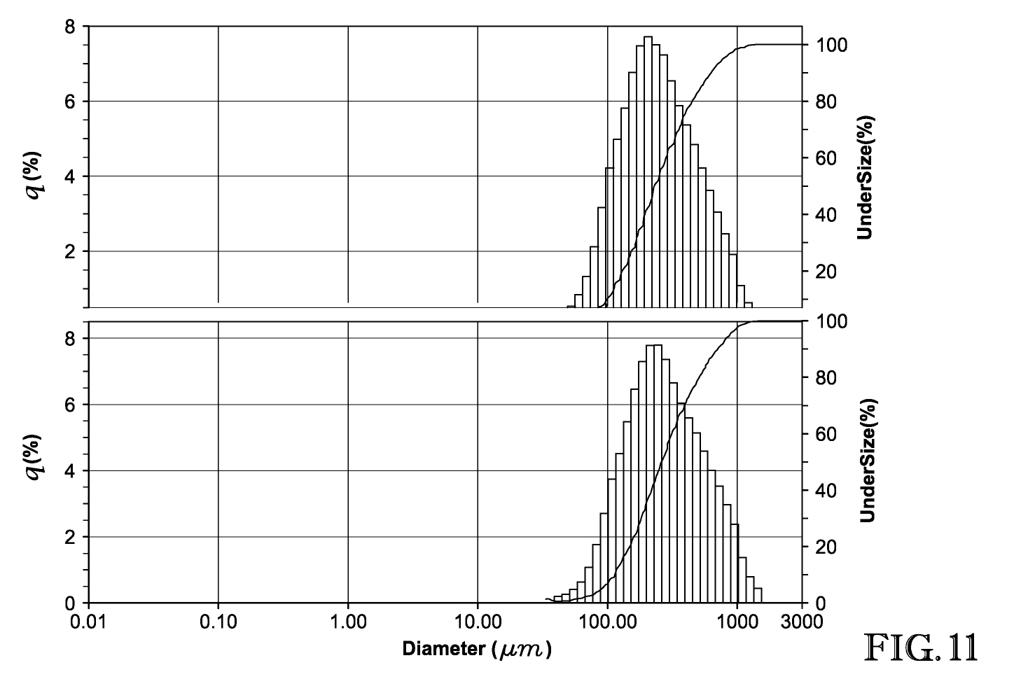

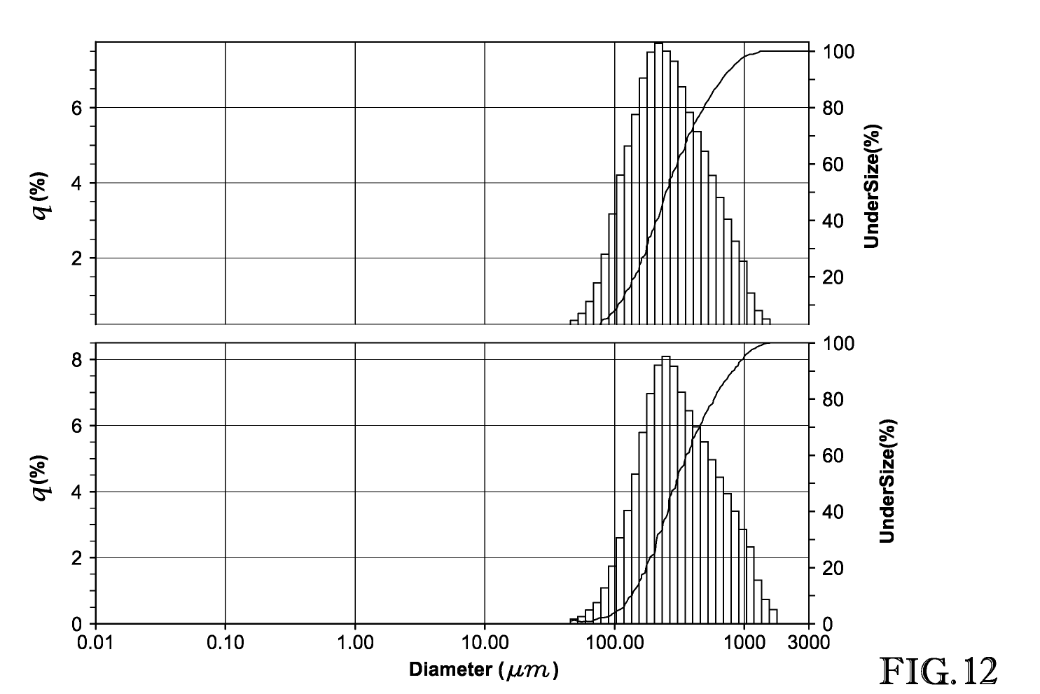

TABLE 5
Select Particle Size Distribution Information for Hydroquinone
Particles Crystallized according to Inventive Cooling Profiles
3-5 and Comparative Cooling Profiles B & C

| | Cooling | PSD | Dv10, | Dv50, | Dv90, |
|---|---|---|---|---|---|
| Sample | Profile | Graph | μm | μm | μm |
| Inventive 3 | FIG. 7 | FIG. 10 | 124.1 | 278.1 | 770.2 |
| | | (lower) | | | |
| Inventive 4 | FIG. 8 | FIG. 11 | 113.4 | 263.7 | 710.9 |
| | | (lower) | | | |
| Inventive 5 | FIG. 9 | FIG. 12 | 132.2 | 295.9 | 807.2 |
| | | (lower) | | | |
| Comparative B | FIG. 6 | FIGS. 10-12 | 106.7 | 245.3 | 651.2 |
| | | (upper) | | | |
| Comparative C | Not | FIG. 13 | 135.9 | 341.9 | 823.6 |
| | shown | (lower) | | | |

### Example 4

Seven separate batches of hydroquinone-containing solution were cooled and crystallized in a hydroquinone production plant. Each of the seven batches was cooled by evaporative cooling according to a profile similar to Inventive Profile 1 shown in FIG. 5. More specifically, during each of the seven trials, a hydroquinone-containing solution was cooled from an initial temperature to a crystallization temperature at a first average cooling rate, r_{C1}, of 0.01167° C./s. Once the solution reached a temperature of 67° C. (or, upon onset of crystallization if visual detection was used), the temperature was held constant for a period of 30 minutes. After the initial hold period, the crystallization slurry was cooled at a rate of 0.00253° C./s to a temperature of 50° C., at which point the temperature of the slurry was maintained at 50° C. for a total of 30 minutes. After the completion of the crystallization hold period, the slurry was cooled at a rate of 0.00278° C./s to a final temperature of 15° C.

The temperature of the solution was controlled manually for four of the batches (Trials 1 through 4) and automatically, via a distributed control system (DCS), for the remaining three (Trials 5 through 7). In addition, the onset of crystallization for each of Trials 1 through 4 was determined visually, while no visual detection was used for Trials 5 through 7. Four additional batches (Trials A through D) of hydroquinone-containing solution were also cooled according to Comparative Profile A shown in FIG. 5 and described in Example 1.

Upon cooling to the final temperature, the crystallized particles in the final slurry in each batch were separated from the liquid phase and dried, and the particle size distribution of the resulting dried particles was determined as described previously. The results of the particle size distribution analyses for each of Trials A through D and Trials 1 through 7 are summarized in Table 6, below.

TABLE 6
Particle Size Distribution of Several Hydroquinone Samples

| | | Visual | | | | |
|---|---|---|---|---|---|---|
| Trial | Type | Detection? | Dᵥ10 | Dᵥ50 | Dᵥ90 | Span |
| Trial A | Comparative | No | 108 | 253 | 644 | 2.12 |
| Trial B | Comparative | No | 116 | 268 | 684 | 2.12 |
| Trial C | Comparative | No | 111 | 264 | 686 | 2.18 |
| Trial D | Comparative | No | 111 | 262 | 677 | 2.16 |
| Trial 1 | Inventive | Yes | 139 | 316 | 815 | 2.14 |
| Trial 2 | Inventive | Yes | 142 | 317 | 830 | 2.17 |
| Trial 3 | Inventive | Yes | 140 | 318 | 849 | 2.23 |
| Trial 4 | Inventive | Yes | 144 | 327 | 830 | 2.10 |
| Trial 5 | Inventive | No | 140 | 321 | 819 | 2.12 |
| Trial 6 | Inventive | No | 137 | 317 | 821 | 2.16 |
| Trial 7 | Inventive | No | 139 | 319 | 887 | 2.34 |

### Example 5

An approximately 100-gram sample of hydroquinone particles from Trial 5 in Example 4 (“Inventive Sample 1”) was combined with approximately 2 to 3 mL of immersion oil (commercially available from Electron Microscopy Sciences, Catalog #16916-01) to form an oil-particle dispersion. A single drop of the oil-particle dispersion was placed on a glass microscope slide and covered with a glass slip. The resulting slide was examined under a Nikon 90i compound optical microscope in the bright-field mode using a 2× Objective lens. Any slides that, upon inspection, exhibited clumped or otherwise unseparated hydroquinone particles were discarded and a new slide was prepared for analysis as described above.

Under the microscope, ten total images of the sample were captured using NIS-Elements BR 2.30 image capture and analysis software by Nikon. The various images focused on different areas of the sample in order to capture as much of the sample as possible. Using the software that had been calibrated to the magnification used when capturing the images, the major dimension (length) and minor dimension (width) of each of the particles present in each image were then analyzed. Any particles partially-imaged (i.e., present at the edge of the field of view and “cut off” by the placement of the image) were ignored. In total, about 400 individual particles were analyzed and the data was exported from the image software to a spreadsheet. In the spreadsheet, aspect ratios for each of the measured particles were calculated by dividing the major dimension (length) of each particle by its minor dimension (width). The above procedure was repeated for a representative sample of hydroquinone particles from two conventionally-prepared batches, referred to as Comparative Samples E and F.

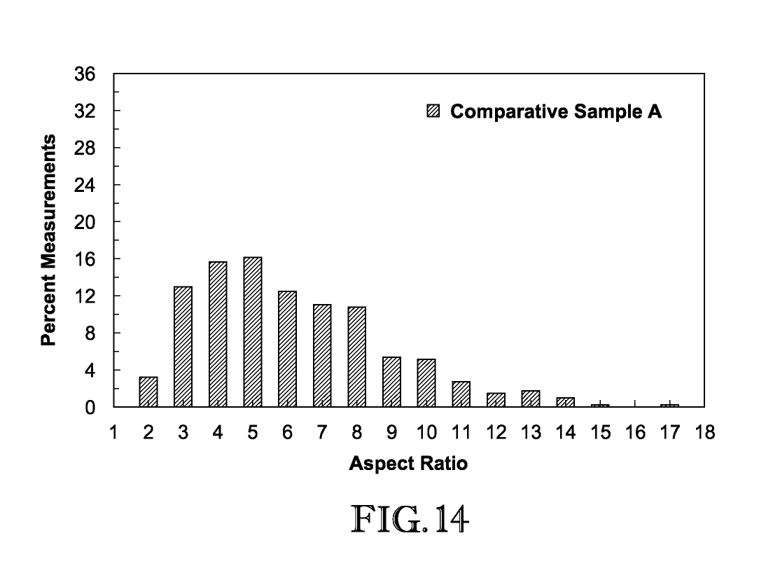

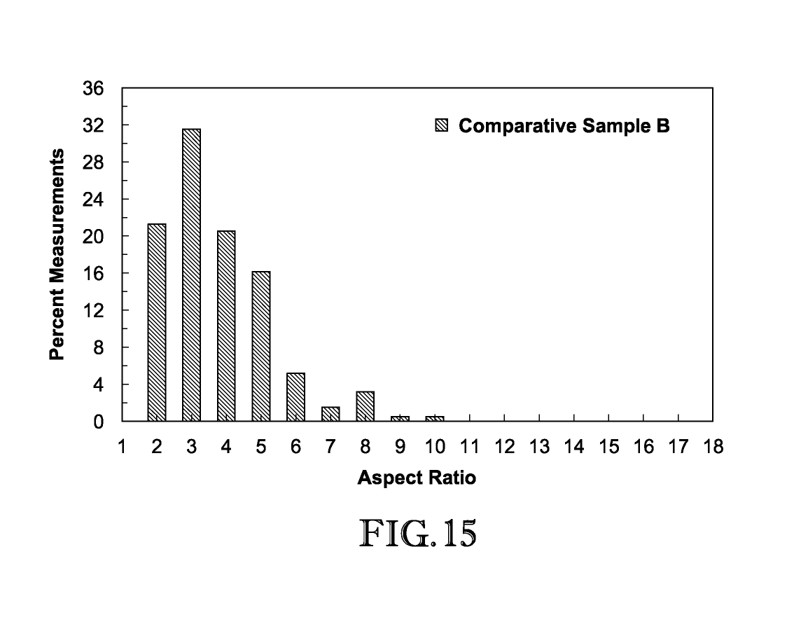

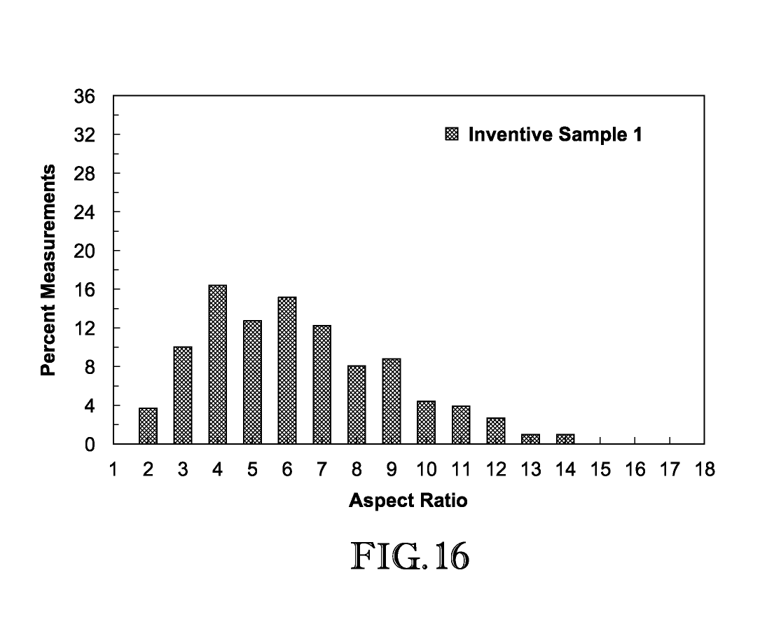

A number-averaged aspect ratio distribution was constructed, using a bin size of 1, for each of Comparative Sample E, Comparative Sample F, and Inventive Sample 1. These distributions are shown in FIGS. 14 through 16, respectively. A statistical analysis of each of these distributions was performed, and values for the tenth percentile aspect ratio (A10), median aspect ratio (A50), the ninetieth percentile aspect ratio (A90), and the span (Span_{A}) for each distribution shown in FIGS. 14 through 16 are summarized in Table 7, below. Additionally, number-averaged length distributions for each of the samples were also constructed, and the tenth percentile length (L10), the median length (L50), the ninetieth percentile length (L90), and the span (Span_{L}) for each of the length distributions is summarized in Table 8, below.

TABLE 7
Results of Statistical Analysis of Aspect Ratio Distributions

| Sample | A50 | A10 | A90 | Span_{A} |
|---|---|---|---|---|
| Comparative E | 5.21 | 2.56 | 9.47 | 1.33 |
| Comparative F | 2.91 | 1.69 | 5.06 | 1.16 |
| Inventive 1 | 5.38 | 2.85 | 9.69 | 1.27 |

TABLE 8
Results of Statistical Analysis of Length Distributions

| Sample | L50 | L10 | L90 | Span_{L} |
|---|---|---|---|---|
| Comparative E | 368 | 134 | 786 | 1.77 |
| Comparative F | 235 | 100 | 537 | 1.86 |
| Inventive 1 | 510 | 170 | 1071 | 1.77 |

The preferred forms of the invention described above are to be used as illustration only and should not be used in a limiting sense to interpret the scope of the present invention. Obvious modifications to the exemplary embodiments, set forth above, could be readily made by those skilled in the art without departing from the spirit of the present invention. The inventors hereby state their intent to rely on the Doctrine of Equivalents to determine and assess the reasonably fair scope of the present invention as pertains to any apparatus not materially departing from but outside the literal scope of the invention as set forth in the following claims.

1. A process for producing crystallized hydroquinone, said process comprising:
(a) providing a hydroquinone-containing solution having an initial temperature, T₁;
(b) cooling said solution from T₁ to a first intermediate temperature, T₂, to thereby provide a cooled solution, wherein said cooling is performed at a first average cooling rate, r_{C1};
(c) further cooling said cooled solution from T₂ to a second intermediate temperature, T₃, to thereby provide a further cooled solution, wherein said further cooling is performed at a second average cooling rate, r_{C2}, wherein r_{C2} is less than r_{C1}; and
(d) subsequent to said further cooling, crystallizing some of the hydroquinone in at least a portion of said further cooled solution to thereby provide crystallized hydroquinone.

2. The process of claim 1, wherein the ratio of r_{C1} to r_{C2} (r_{C1}:r_{C2}) is in the range of from 2:1 to 25:1.

3. The process of claim 1, wherein T₁ is in the range of from 70° C. to 100° C. and T₂ is in the range of from 65° C. to 77° C.

4. The process of claim 1, wherein said hydroquinone-containing solution is an aqueous solution.

5. The process of claim 1, further comprising, during said crystallizing of step (d), maintaining the temperature of said further cooled solution at a substantially constant temperature for a period of time in the range of from 10 minutes to 90 minutes.

6. The process of claim 1, wherein said crystallizing of step (d) includes crystallizing at least 30 weight percent of the hydroquinone originally present in said hydroquinone-containing solution.

7. The process of claim 1, wherein said hydroquinone-containing solution comprises hydroquinone in an amount in the range of from 1 weight percent to 60 weight percent, based on the total weight of said solution.

8. The process of claim 1, further comprising, prior to said crystallizing of step (d), additionally cooling said further cooled solution from T₃ to a third intermediate temperature, T₄, to thereby provide an additionally cooled solution, wherein said additional cooling is performed at a third average cooling rate, r_{C3}, wherein r_{C3} is less than r_{C2}, and wherein said hydroquinone is crystallized during said crystallizing of step (d).

9. The process of claim 8, wherein the ratio of r_{C2} to r_{C3} (r_{C2}:r_{C3}) is in the range of from 2:1 to 25:1 and wherein the ratio of r_{C1} to r_{C3} (r_{C1}:r_{C3}) is in the range of from 10:1 to 100:1.

10. The process of claim 1, further comprising, subsequent to said crystallizing of step (d), separating at least some liquid from said crystallized hydroquinone to thereby provide wet hydroquinone crystals and drying said wet hydroquinone crystals to thereby provide dried hydroquinone particles, wherein the maximum temperature achieved during said drying is in the range of from 65° C. to 105° C.

11. The process of claim 10 further comprising, subsequent to said drying, cooling said dried hydroquinone particles to thereby provide cooled hydroquinone particles, wherein said cooling reduces the temperature of said dried hydroquinone particles to a temperature less than 60° C., and further comprising, packaging said cooled hydroquinone particles in at least one shipping container.

12. The process of claim 11, wherein said cooled hydroquinone particles have a particle size distribution (PSD) having a median particle size in the range of from 280 microns to 335 microns and a span of at least 2.1.

13. A process for producing crystallized hydroquinone, said process comprising:
(a) cooling a hydroquinone-containing solution in a crystallization zone to form crystallized hydroquinone;
(b) during at least a portion of said cooling of step (a), measuring the value of at least one direct crystallization parameter in said crystallization zone to thereby provide a measured value;
(c) comparing said measured value of said crystallization parameter to a threshold value for that crystallization parameter to determine a difference;
(d) based on said difference, adjusting said cooling of step (a) to thereby cause at least a portion of said hydroquinone to crystallize out of said solution.

14. The process of claim 13, wherein said direct crystallization parameter is selected from the group consisting of normalized particle count, total particle count, particle development rate, and combinations thereof.

15. The process of claim 14, wherein said direct crystallization parameter measured during step (b) comprises particle development rate and wherein said threshold value used in said comparing of step (c) is a minimum threshold value in the range of from 0.5 particles per minute (particles/min) to 5 particles/min and wherein said adjusting of step (d) includes reducing or stopping said cooling of step (a).

16. The process of claim 15, further comprising, after said adjusting of step (d), further cooling said solution to a final crystallization temperature to thereby provide a crystallization slurry comprising a liquid portion and a plurality of hydroquinone crystals and drying at least a portion of said hydroquinone crystals to thereby provide dried hydroquinone particles.

17. The process of claim 16, further comprising, cooling at least a portion of said dried hydroquinone particles to thereby provide cooled hydroquinone particles, wherein said cooling and said drying are carried out in separate vessels.

18. The process of claim 13, wherein said measuring of step (b) includes visually monitoring said direct crystallization parameter.

19. The process of claim 18, wherein said visually monitoring is performed over a measurement area within said crystallization zone, wherein said measurement area has an area of at least about 10 cm², and wherein at least a portion of said visually monitoring comprises use of visual imaging software and equipment.

20. The process of claim 13, wherein at least a portion of steps (b) through (d) are carried out with an automated control system.

21. A process for producing a plurality of hydroquinone particles, said process comprising:
(a) crystallizing a hydroquinone-containing solution in a crystallization zone to thereby provide a crystallization fluid and a plurality of crystallized particles;
(b) separating at least a portion of said crystallization fluid from said crystallized particles in a separation zone to thereby provide separated hydroquinone crystals;
(c) drying at least a portion of said separated hydroquinone crystals in a drying zone to thereby provide dried crystalline particles;
(d) cooling at least a portion of said dried crystalline particles in a cooling zone to thereby provide a plurality of cooled particles, wherein at least 50 volume percent of said cooled particles have a temperature of less than 80° C.; and
(e) transporting at least a portion of said cooled particles into a loading zone and loading said cooled particles into at least one storage container.

22. The process of claim 21, wherein at least a portion of said cooling of step (d) and said transporting of step (e) are performed simultaneously.

23. The process of claim 21, wherein said drying of step (c) includes heating said crystals to a maximum temperature in the range of from 65° C. to 105° C. and wherein said cooled particles have a temperature of less than 60° C.

24. The process of claim 21, wherein the temperature of said cooled particles is measured with a direct temperature indicator.

25. The process of claim 21, wherein a representative sample of said cooled particles has a volume-averaged particle size distribution having a Dv50 of less than 340 microns.

26. The process of claim 25, further comprising, subsequent to said transporting of step (e), storing and/or transporting said cooled particles in said storage container for a storage period of at least 5 days and, after said storage period, unloading the particles from said storage container to thereby provide unloaded particles, wherein said unloaded particles have substantially the same particle size distribution as said cooled particles loaded into said storage container in step (e).

27. The process of claim 21, wherein said separating of step (b) includes removing at least a portion of said crystallized fluid from said crystallized particles and washing said crystallized particles with an aqueous wash liquid to provide said separated hydroquinone crystals.

28. The process of claim 21, wherein said storage container, when loaded with said cooled hydroquinone particles, has a mass of at least 100 kg.

29. The process of claim 21, wherein said transporting of step (e) includes transporting at least 500 kg/hour of said cooled particles into said loading zone.

30. A process for producing crystallized hydroquinone, said process comprising:
(a) cooling a hydroquinone-containing slurry from a first crystallization temperature, T_{C1}, to a second crystallization temperature, T_{C2}, in a crystallization zone;
(b) maintaining the temperature of said hydroquinone-containing slurry within about 5° C. of T_{C2} for a crystallization hold period, wherein T_{C2} is less than 60° C.; and
(c) subsequent to said crystallization hold period, further cooling said hydroquinone-containing slurry from a third crystallization temperature, T_{C3}, to a final crystallization temperature, T_{CF}, in said crystallization zone.

31. The process of claim 30, wherein T_{C2} is in the range of from 35° C. to 55° C.

32. The process of claim 30, wherein said crystallization hold period is maintained for a length of time in the range of from 15 minutes to 1.5 hours.

33. The process of claim 30, wherein T_{C2} is in the range of from 45° C. to 55° C. and wherein said crystallization hold period is maintained for a length of time in the range of from 30 minutes to 1 hour.

34. The process of claim 33, wherein T_{C1} is in the range of from 55° C. to 85° C., wherein T_{C3} is in the range of from 45° C. to 55° C., and wherein T_{CF} is in the range of from 5° C. to 30° C., wherein said cooling of step (a) is carried out for a first cooling period having a length of time from 1 hour to 8 hours, and wherein said further cooling of step (c) is carried out for a second cooling period having a length of time from 3 hours to 10 hours.

35. The process of claim 30, wherein said cooling of step (a) is carried out at a first average crystallization cooling rate, R_{C1}, and wherein said further cooling of step (c) is carried out at a second average crystallization cooling rate, R_{C2}, wherein the ratio of R_{C1} to R_{C2} (R_{C1}:R_{C2}) is in the range of from 0.5:1 to 2:1.

36. The process of claim 35, wherein R_{C1}:R_{C2} is less than 1:1.

37. The process of claim 30, further comprising prior to said cooling of step (a), maintaining the temperature of said hydroquinone-containing slurry at a temperature within about 5° C. of T_{C1} for an initial hold period of at least 15 minutes.

38. The process of claim 37, wherein said cooling of step (a) is carried out for a first crystallization cooling period and wherein the ratio of the length of time of said initial hold period to the length of time of said first crystallization cooling period is in the range of from 0.10:1 to 1:1.

39. The process of claim 37, wherein the ratio of the length of time of said crystallization hold period to the length of time of said initial hold period is in the range of from 0.5:1 to 2:1.

40. The process of claim 30, wherein said maintaining of step (b) includes maintaining the temperature of said hydroquinone-containing slurry within about 2° C. of T_{C2}.

41. The process of claim 30, wherein at least a portion of said cooling of step (a) and/or at least a portion of said cooling of step (c) are performed at a pressure below atmospheric pressure.

42. The process of claim 30, wherein T_{C1} is in the range of from 60° C. to 75° C., wherein said maintaining of step (b) includes maintaining the temperature of said hydroquinone-containing slurry within about 2° C. of T_{C2}, wherein said crystallization hold period is maintained for a length of time in the range of from 15 minutes to 1 hour, wherein T_{C2} and T_{C3} are in the range of from 45° C. to 55° C., wherein T_{CF} is in the range of from 5° C. to 27° C., wherein said cooling of step (a) is carried out for a first cooling period having a length of 1 hour to 8 hours, and wherein said further cooling of step (c) is carried out for a second cooling period having a length of from 2 hours to 10 hours.

43. The process of claim 30, further comprising subsequent to said further cooling of step (c), separating at least some liquid from said hydroquinone-containing slurry to thereby provide wet hydroquinone crystals; drying said wet hydroquinone crystals to thereby provide dried hydroquinone particles, wherein the maximum temperature achieved during said drying is in the range of from 65° C. to 105° C.; and packaging said dried hydroquinone particles in at least one shipping container, wherein a representative sample of said dried hydroquinone particles has a volume-averaged particle size distribution having a Dv50 of at least 285 microns and a span of at least 2.05.

44. The process of claim 30, wherein at least a portion of steps (a) through (c) are performed using an automated control system.

45. A process for producing crystallized hydroquinone, said process comprising:
(a) cooling a hydroquinone-containing solution from an initial temperature, T₁, to a crystallization temperature, T_{C}, in a crystallization zone;
(b) crystallizing at least a portion of said hydroquinone in said solution to provide a crystallized hydroquinone slurry, wherein at least a portion of said crystallizing includes maintaining the temperature of the crystallization zone within about 5° C. of T_{C};
(c) subsequent to said crystallizing, further cooling said crystallized hydroquinone slurry to a final temperature, T_{CF}, over a period of time, Δt, wherein the temperature of said crystallized hydroquinone slurry at a time, t, after initiation of said further cooling of step (c) is within 5° C. of a predicted temperature, Tₚ, provided by the following equation:
Tₚ=T_{C}+λt³,
wherein the crystallization coefficient, λ, is defined by the following equation:
$$λ = ( \frac{{T}_{CF} - {T}_{C}}{Δ {t}^{3}} ) .$$

46. The process of claim 45, wherein T_{C} is in the range of from 65° C. to 68° C.

47. The process of claim 45, wherein Δt is in the range of from 2 hours to 8 hours.

48. The process of claim 45, wherein T_{CF} is in the range of from 5° C. to 30° C.

49. The process of claim 45, further comprising subsequent to said further cooling of step (c), separating some of the crystallized hydroquinone from said crystallized hydroquinone slurry to provide separated hydroquinone particles;
drying at least a portion of said separated hydroquinone particles to provide dried particles; and loading at least a portion of said dried hydroquinone particles into at least one storage container.

50. A composition comprising at least 5 kg of crystallized hydroquinone particles, wherein a representative sample of particles from said composition has a volume-averaged particle size distribution having a Dv50 of at least 285 microns and a span of at least 2.05.

51. The composition of claim 50, wherein the bulk density of said representative sample of particles is at least 0.50 g/cm³.

52. The composition of claim 50, wherein the specific surface area of said representative sample of particles is not more than 0.065 m²/g.

53. The composition of claim 50, wherein said representative sample of particles from said composition has a volume-averaged particle size distribution having a Dv50 of at least 295 microns.

54. The composition of claim 50, wherein said representative sample of particles from said composition has a volume-averaged particle size distribution having a span of at least 2.10.

55. The composition of claim 50, wherein said representative sample of particles from said composition has a volume-averaged particle size distribution having a Dv50 in the range of from 295 to 350 microns and a span in the range of from 2.10 to 2.35.

56. The composition of claim 50, wherein said composition comprises at least 100 kg of said crystallized hydroquinone particles.

57. The composition of claim 50, wherein said composition comprises at least about 1000 kg of said crystallized hydroquinone particles.

58. The composition of claim 50, wherein said composition comprises at least 50 by weight of said crystallized hydroquinone particles, based on the total weight of said composition.

59. A shipping container comprising a space that contains the composition recited in claim 50.

60. A composition comprising at least 5 kg of crystallized hydroquinone particles, wherein a representative sample of particles from said composition has a volume-averaged particle size distribution having a Dv50 of at least 285 microns, wherein said representative sample has a number-averaged aspect ratio distribution having a median aspect ratio (A50) of at least 3:1 and/or a number-averaged length distribution having a median particle size (L50) of at least 250 microns.

61. The composition of claim 60, wherein said A50 is at least 5.25:1.

62. The composition of claim 60, wherein said L50 is at least 375 microns.

63. The composition of claim 60, wherein said particle size distribution has a span of at least 2.05.

64. The composition of claim 60, wherein said A50 is at least 3:1 and said L50 is at least 250 microns.

65. The composition of claim 60, wherein said composition comprises at least 100 kg of said crystallized hydroquinone particles.

66. A shipping container comprising a space that contains the composition recited in claim 60.
